(19) United States
(12) Patent Application Publication
(10) Pub. No.: US 2006/0058346 A1
(43) Pub. Date: Mar. 16, 2006

(54) Novel enantiomers of etrahydroisoquinoline derivatives and theirpharmaceutically acceptable salts, their preparations and pharmaceutical compositions

(76) Inventors: Hye-Sook Yun-Choi, Seoul (KR); Ki Churl Chang, Kyungsannam-do (KR)

Correspondence Address: LUCAS & MERCANTI, LLP, 475 PARK AVENUE SOUTH, 15TH FLOOR, NEW YORK, NY, 10016, US

(21) Appl. No.: 10/513,383

(22) PCT Filed: Oct. 5, 2002
(86) PCT No.: PCT/KR02/01859

§ 371 Date: Nov. 3, 2004

(30) Foreign Application Priority Data
May 7, 2002 (KR) PCT KR02 00846

(51) Int. Cl. C07D 217/00; A61K 31/47

(52) U.S. Cl. 514/310; 546/150

(57) ABSTRACT

The disclosure concerns novel enantiomers of tetrahydroisoquinoline derivatives and their pharmaceutically acceptable salts, their preparations and pharmaceutical compositions. The enantiomers of tetrahydroisoquinoline derivatives are provided which are useful in stimulating heart rate and hypotensive activity, inhibitory activity against platelet aggregation, and suppressive against inducible NO synthase. The enantiomers of tetrahydroisoquinoline derivatives and their pharmaceutically acceptable salts are effective for treating congestive heart failure, hypertension, thrombosis, inflammation, septicemia, cardiac insufficiency, and disseminated intravascular coagulopathy.

### TECHNICAL FIELD

The present invention relates, in general, to novel enantiomers of tetrahydroisoquinoline derivatives and pharmaceutically acceptable salts thereof, preparations and uses thereof. More specifically, the present invention is directed to a novel tetrahydroisoquinoline based enantiomer and a pharmaceutically acceptable salt thereof, characterized in that on the basis of R,S-configurations of the enantiomer, and S-configuration is superior to R-configuration in inducing vasodilation, positive inotropic and chronotropic action, and inhibiting against inducible NO synthetase (hereinafter, abbreviated to “iNOS”) expression as well as against platelet aggregation. On the other hand, R-configuration enantiomer, although the effects are less potent than those of S-configuration enantiomer, is selectively effective in inducing vasodilation, inhibiting against platelet aggregation, and suppressing against iNOS with only very mild cardiotonic effects; A preparation method and a pharmaceutical use thereof.

### BACKGROUND OF THE INVENTION

Of tetrahydroisoquinoline (hereinafter, abbreviated to “THI”) based compounds comprising a ring-closed N-alkylphenylethylamine, 6,7-dihydroxytetrahydroisoquinoline has a fundamental backbone of catecholamine in the chemical structure thereof. Generally represented by epinephrine, norepinephrine and dopamine, catecholamine has 3,4-dihydroxyphenylethylamine as its structural backbone. It is thus reported that many THI based compounds having affinity for various adrenergic α- or β-receptors, depending on the kinds and the positions of substituents, thereby exhibit various pharmacological actions, with agonistic or antagonistic effects.

In particular, THI based compounds, which possess benzyl groups substituted with OH, OCH₃ and halogen on carbon of 1-position, are reported to have strong functions such as bronchodilation, inhibition against platelet aggregation, calcium channel blocking, etc (King, V. F. et al., J. Biol. Chem., 263, 2238-2244, 1988; Triggle, D. J. et al., Med. Res. Rev., 9, 123-180, 1989; Lacorix, P. et al., Eur. J. Pharmacol., 192, 317-327, 1991; Chang, K. C. et al.,. Life. Sci., 51, 64-74, 1992; Chang, K. C. et al., Eur. J. Pharmacol., 238, 51-60, 1993).

Higenamine is a THI compound, of which 4-hydroxybenzyl group is attached to 1-carbon position and also two hydroxyl groups are attached to 6-position and 7-position. The said compound is structurally similar to dobutamine used as cardiotonic agent in clinical trial. Higenamine is found to have effects such as increase of contractile force and heart rates in excised heart, in-vitro effect as inhibitory effect against platelet aggregation, increase of cardiac output, hypotension or inhibitory effect against platelet aggregation in rat or rabbit. Also, we found that higenamine had effects such as decrease of expression of iNOS following decrease of production of excess nitric oxide (NO), inhibitory effect against decreased vascular responsiveness by LPS, or decrease of mortality caused by endotoxin. Then, we applied to Korean institute of patent organization in 1993-4-26. The said application was admitted (Korea patent, No. 110506). The said contents were published in learned circles. (Chang, K. C. et al., Can. J. Physiol. Pharmacol., 72, 327-334, 1994; Yun-Choi, H. S. et al., Yakhak Hoeju, 38, 191-196, 1994; Kang, Y. J. et al., Kor. J. Physio. Pharmacol. 1, 297-302, 1997; Shin, K. H. et al., Natural Products Sciences, 2, 24-28, 1996). However, higenamine had disadvantage such as shortness of pharmaceutical duration or slightness of the said effects.

We searched for potent agents that were derived from higenamine-modifed structure with excellent pharmacological effects. Then, we found that the said materials had potent in vitro effects such as increasing contractile forces and beat rates in heart excised from rats and vasodilating in isolated blood vessel contracted by phenylephrine and in vivo effects such as increasing heart rates and lowering blood pressure in rabbit to which the said material was administered. The effects of the said materials were similar to higenamine. Then, we applied to Korean institute of patent organization in 1994-12-1. The said application was admitted (Korea patent, No. 148755). The said contents were published in learned circles. (Lee, Y. H. et al., Life Sciences, 55, 415-420, 1994, Chong W. S. et al., Kor. J. Physiol. Pharmacol. 2, 323-330, 1998, Chang K. C. et al., Kor. J. Physiol. Pharmacol. 2, 461-469, 1998).

Meanwhile, as examples of substances applicable for use in treatment of congestive heart failure, there are cardiac glycoside such as digoxin and digitoxin, adrenergic β-agonists, such as dopamine and dobutamine, vasodilating agents such as angiotensin-converting enzyme inhibitors, angiotensin-receptor antagonists and Ca²⁺ channel antagonists or phosphodiesterase inhibitors and diuretics. But such substances exhibit undesirable side-effects, such as arrhythmia, excessive contraction of heart and low blood pressure, and are thus limited in their use. Therefore, there is need for a therapeutic agent for treatment of heart failure having a novel mechanism, different from the functions of digitalis, dopamine, angiotensin converting enzyme inhibitor etc. stated above. Administration of cardiotonic agent increasing the heart contractile force with a drug relieving the burden of heart by inhibiting a formation of thrombus following smoothly flowing blood in blood vessel resulted in good treatment effect in heart failure patient. Therefore, cardiotonic agent such as digoxin and digitoxin or dopamine is administered in combination with vasodilator (hypotension) and/or platelet-aggregation inhibitor to increase treatment effect. However, combined administration of various drugs, because of the probable interactions of the respective drugs affects absorption and metabolism of each drug, increases the risk such as side-effect and the said risk limits the use of the drug. According to recent research, in the case of heart failure, it is found that TNF-α or iNOS expression level is higher in blood and tissues. Hence, in these days, it is known that drugs directly stimulating myocardial contraction are favorable for short-term use, and the beta-blocking agents, which have been contraindicated, affect remodeling of the heart and are profitable for use over a long-term period. Furthermore, as particular knowledge related to diseases is generalized day-by-day, conventional therapeutics are also changing.

Tetrahydroisoquinolines are alkaloids present in nature, and are exemplified by papaverin or higenamine. Such alkaloids have a variety of pharmacological functions. From recent investigation, as 1-α-naphthylmethyl-6,7-dihydroxy-1,2,3,4-tetrahydroisoquioline and 1-β-naphthylmethyl-6,7-tetrahydroisoquinoline synthesized by modifying the structure of natural higenamine, have been found to have complex and simultaneous effects such as cardiotonic activity, vasodilation (hypotension), platelet-aggregation inhibition, suppressive activity against iNOS, they are suggested to be useful as therapeutic agent for heart failure, thrombosis, tissue damage induced by the iNOS expression following excessive production of NO, septicemia and disseminated intravascular coagulation. Then, we applied to Korean institute of patent organization (Korea patent No. 352425 PAT/KR99/00631).

Although in korea patent No. 110506, 148755 and 352425, racemic mixtures corresponding to the compound of present invention were known, definite establishment about the isomers thereof, i.e. optically pure (+) or (−) optical isomers were not existed, and preparation and separation thereof, pharmaceutical effects of respective optical isomers and correlation between racemic mixture and the said optical isomers were not referred at all.

Generally, two optically pure compounds that are in mirror image-relationship to one another possess the same physical properties, except one-optical activity. In detail, the two enantiomers are completely or almost identical in, for example, melting point, boiling point, solubility, density and refractive index, but completely opposite in optical rotation. Since the two enentiomers rotate the plane of polarized light in equal but opposite directions, no net optical rotation is observed when they are mixed. In other words, the optical rotation of a racemate is zero in theory and near zero in practicability. Discrepancy in physiological activity and toxicity of racemic mixture and respective enantiomers occurred by the said difference in rotation, i.e. the difference in arrangement of substitutents of chiral carbon. However, consistent relation was not existed in the said discrepancy. Therefore, it is impossible to predict the said relation from the conservative results. For example, ofloxacin is recemic mixture, (−)-enantiomer thereof is levofloxacin. Antibiotic activity of levofloxacin was two times as excellent as ofloxacin, and 8-128 times as excellent as (+)-ofloxacin i.e. the other enantiomer of ofloxacin (Drugs of the future. 17(7): 559-563 (1992)). Also, (±)-cisapride, racemic mixture had toxicity in co-administration with other drug. However, (+)-norcisapride, optical isomer of cisapride had no toxicity in the said administration. Consequently, the said result showed that (−)-cisapride is toxic optical isomer. (Stephen C. Stinson, Chemical & Engineering News, 76(3), 3 (1998)). Also, in Korea patent 179654, cerebral blood flow-simulating activity of R-(−) optical isomer is three times as strong as that of S-(+) optical isomer. And cerebral blood flow-simulating activity of R-(−) optical isomer is lasted three times as long as S-(+) optical isomer. For temafloxacin, it was reported that antibiotic activity and pharmacokinetics for racemic mixture and the optical isomers had no difference (Daniel T. W. Chu, et al., J. Med. Chem., 34, 168-174(1991)). Therefore, it is necessary to resolve pure optical isomers from racemic mixture and investigate their properties, because of the unpredictable physiological differences between optical isomers and the racemic mixture.

As aforementioned, use of the racemic mixture without resolution has the problem, which is that while one enantiomer thereof has excellent pharmaceutical effect and no toxicity, the other enantiomer thereof has toxicity. The said problem was frequently shown in pharmaceutically used compound. Also, use of the racemic mixture without resolution has the bodily burden, which is caused by the administration of one enantiomer with slight pharmaceutical effect in the same amount, during the administration of the other one with excellent effect.

We, the inventors of the present invention, have tried to synthesize each enantiomer of higenamine, 1-α-naphthylmethyl-6,7-dihydroxy-1,2,3,4-tetrahydroisoquioline and 1-β-naphthylmethyl-6,7-dihydroxy-1,2,3,4-tetrahydroisoquinoline represented by formula 1 or formula 2 and to examine pharmacological effects thereof. It was thus found that S-configuration enantiomers are superior to R-configuration enantiomers and racemic mixture, in various pharmaceutical effects. Also, toxicity of S-configuration enantiomers is weaker than that of racemic mixture thereof. On the other hand, it was found that R-configuration enantiomers, although the effects are less potent than those of S-configuration enantiomers, are selectively effective in vasodilating, inhibiting against platelet aggregation, and suppressing against iNOS with only very mild cardiotonic effects.

### SUMMARY OF THE INVENTION

Accordingly, it is an object of the present invention to provide optically active tetrahydroisoquinoline derivatives having properties of enhancement or selectiveness of myocardial contractile force, vasodilation, inhibition against platelet aggregation and suppressive activity against iNOS expression, according to R,S-configurations thereof.

It is another object of the present invention to provide a method of producing such derivatives.

It is a further object of the present invention to provide a therapeutic use of such derivatives for heart failure, hypertension, thrombosis, inflammation, septicemia, DIC, and cardiac insufficiency.

### BEST MODE OF CARRYING THE INVENTION

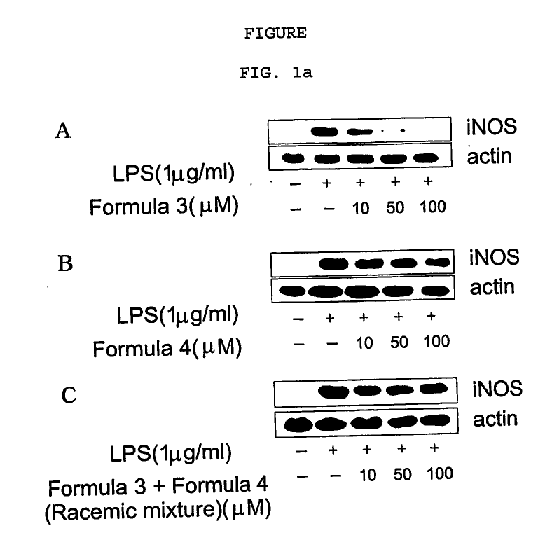

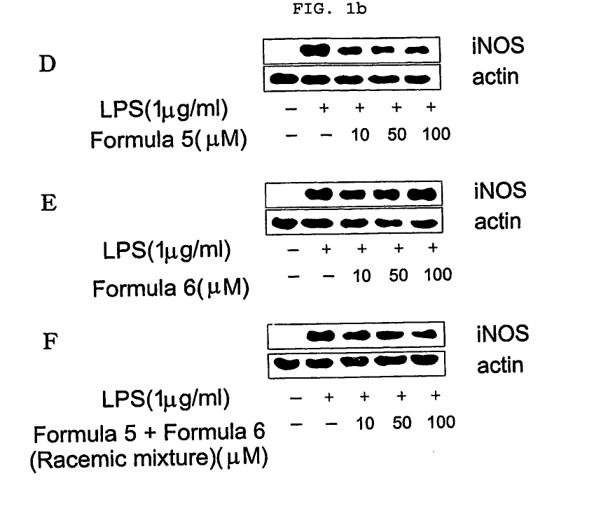

FIG. 1a˜1c show the Western blot analysis for measurement of inhibiting effect on iNOS expression by LPS and IFN-γ in macrophages;
A: LPS (1 μg/mL)+the compound represented by formula 3 (0, 10, 50, 100 μM)
B: LPS (1 μg/mL)+the compound represented by formula 4 (0, 10, 50, 100 μM)
C: LPS (1 μg/mL)+racemic mixture of the compounds represented by formula 3 and 4 (0, 10, 50, 100 μM)
D: LPS (1 μg/mL)+the compound represented by formula 5 (0, 10, 50, 100 μM)
E: LPS (1 μg/mL)+the compound represented by formula 6 (0, 10, 50, 100 μM)
F: LPS (1 μg/mL)+racemic mixture of the compounds represented by formula 5 and 6 (0, 10, 50, 100 μM)
G: LPS (1 μg/mL)+the compound represented by formula 7 (0, 10, 50, 100 μM)
H: LPS (1 μg/mL)+the compound represented by formula 8 (0, 10, 50, 100 μM)
I: LPS (1 μg/mL)+racemic mixture of the compounds represented by formula 7 and 8 (0, 10, 50, 100 μM)

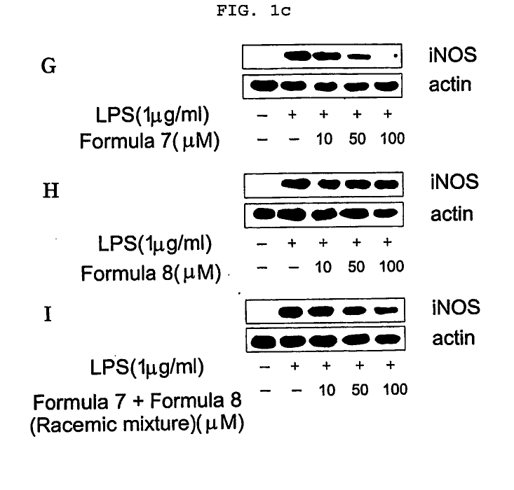

FIG. 2 shows the number of platelets in blood when 15 mg/kg of LPS was intravenously infused to rat for four hours after administration of the compounds represented by formulas 3 to 8.
A: Normal group.
B: Group to which LPS was only administered.
1: Group to which LPS and the compound represented by formula 3 was administered
2: Group to which LPS and the compound represented by formula 4 was administered
3: Group to which LPS and the compound represented by formula 5 was administered
4: Group to which LPS and the compound represented by formula 6 was administered
5: Group to which LPS and the compound represented by formula 7 was administered
6: Group to which LPS and the compound represented by formula 8 was administered

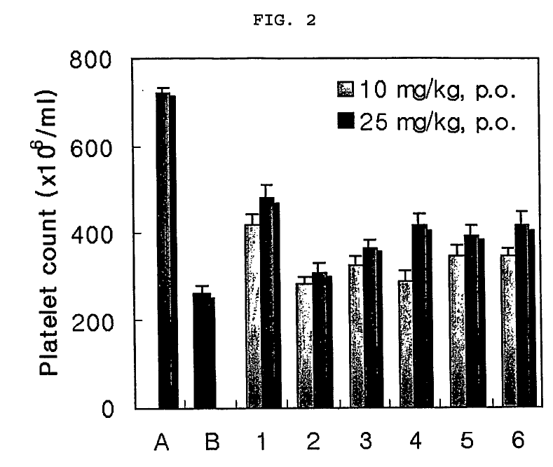

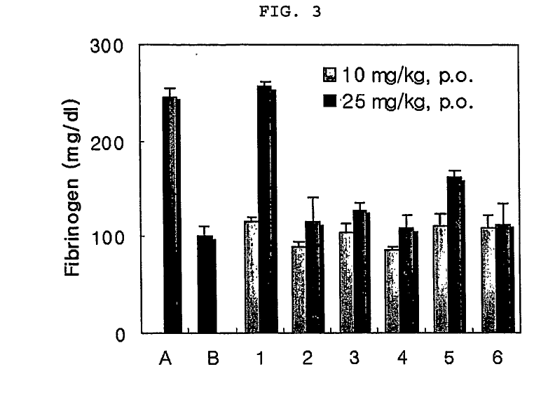

FIG. 3 shows concentration of fibrinogen in blood when 15 mg/kg of LPS was intravenously infused to rat for four hours after administration of the compounds represented by formulas 3 to 8.
A: Normal group.
B: Group to which LPS was only administered.
1: Group to which LPS and the compound represented by formula 3 was administered
2: Group to which LPS and the compound represented by formula 4 was administered
3: Group to which LPS and the compound represented by formula 5 was administered
4: Group to which LPS and the compound represented by formula 6 was administered
5: Group to which LPS and the compound represented by formula 7 was administered
6: Group to which LPS and the compound represented by formula 8 was administered

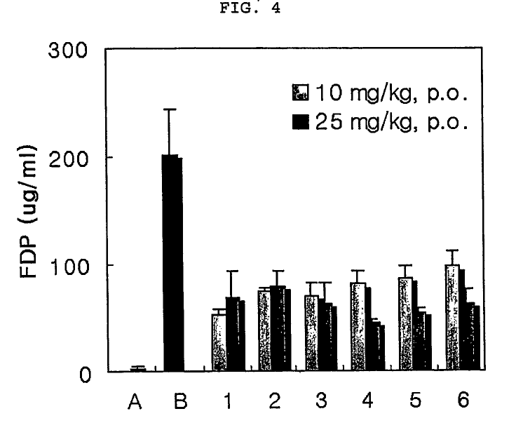

FIG. 4 shows concentration of FDP (fibrine/fibrinogen degradation product) in blood when 15 mg/kg of LPS was intravenously infused to rat for four hours after administration of the compounds represented by formulas 3 to 8.
A: Normal group.
B: Group to which LPS was only administered.
1: Group to which LPS and the compound represented by formula 3 was administered
2: Group to which LPS and the compound represented by formula 4 was administered
3: Group to which LPS and the compound represented by formula 5 was administered
4: Group to which LPS and the compound represented by formula 6 was administered
5: Group to which LPS and the compound represented by formula 7 was administered
6: Group to which LPS and the compound represented by formula 8 was administered

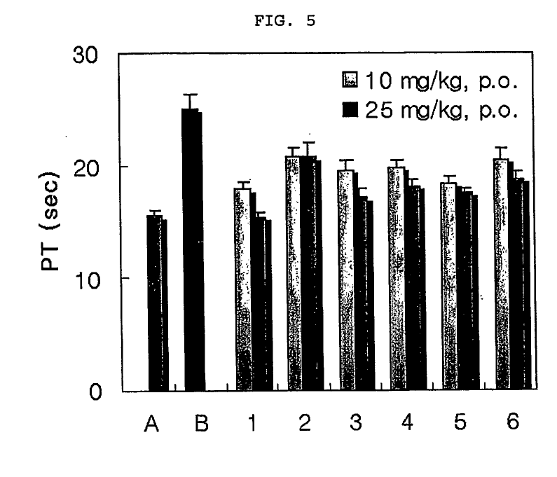

FIG. 5 shows prothrombin time (hereinafter abbreviated into “PT”) when 15 mg/kg of LPS was intravenously infused to rat for four hours after administration of the compounds represented by formulas 3 to 8.
A: Normal group.
B: Group to which LPS was only administered.
1: Group to which LPS and the compound represented by formula 3 was administered
2: Group to which LPS and the compound represented by formula 4 was administered
3: Group to which LPS and the compound represented by formula 5 was administered
4: Group to which LPS and the compound represented by formula 6 was administered
5: Group to which LPS and the compound represented by formula 7 was administered
6: Group to which LPS and the compound represented by formula 8 was administered

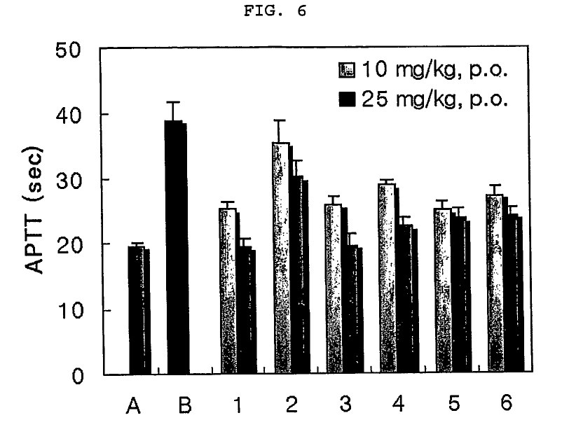

FIG. 6 shows activated partial thromboplastin (hereinafter abbreviated into “APTT”) when 15 mg/kg of LPS was intravenously infused to rat for four hours after administration of the compounds represented by formulas 3 to 8.
A: Normal group.
B: Group to which LPS was only administered.
1: Group to which LPS and the compound represented by formula 3 was administered
2: Group to which LPS and the compound represented by formula 4 was administered
3: Group to which LPS and the compound represented by formula 5 was administered
4: Group to which LPS and the compound represented by formula 6 was administered
5: Group to which LPS and the compound represented by formula 7 was administered
6: Group to which LPS and the compound represented by formula 8 was administered

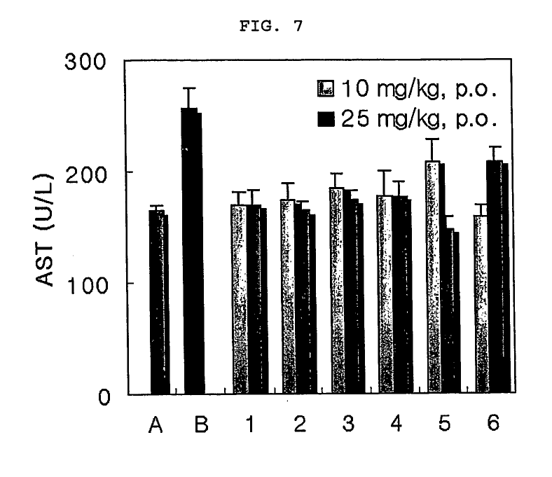

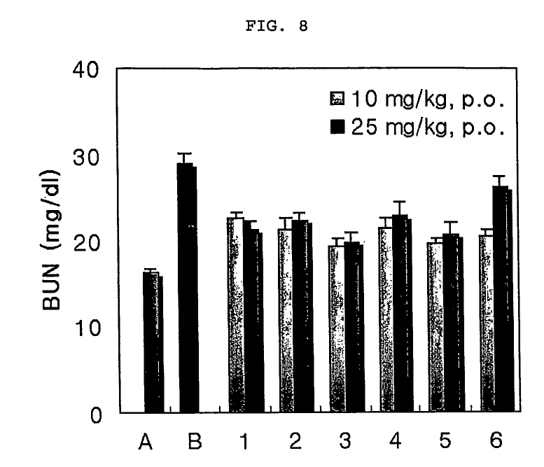

FIG. 7 shows the serum level of serum aspartate amino transferase (hereinafter, abbreviated into “AST”) when 15 mg/kg of LPS was intravenously infused to rat for four hours after administration of the compounds represented by formulas 3 to 8.
A: Normal group.
B: Group to which LPS was only administered.
1: Group to which LPS and the compound represented by formula 3 was administered
2: Group to which LPS and the compound represented by formula 4 was administered
3: Group to which LPS and the compound represented by formula 5 was administered
4: Group to which LPS and the compound represented by formula 6 was administered
5: Group to which LPS and the compound represented by formula 7 was administered
6: Group to which LPS and the compound represented by formula 8 was administered

FIG. 8 shows blood urea nitrogen level when 15 mg/kg of LPS was intravenously infused to rat for four hours after administration of the compounds represented by formulas 3 to 8.
A: Normal group.
B: Group to which LPS was only administered.
1: Group to which LPS and the compound represented by formula 3 was administered
2: Group to which LPS and the compound represented by formula 4 was administered
3: Group to which LPS and the compound represented by formula 5 was administered
4: Group to which LPS and the compound represented by formula 6 was administered
5: Group to which LPS and the compound represented by formula 7 was administered
6: Group to which LPS and the compound represented by formula 8 was administered

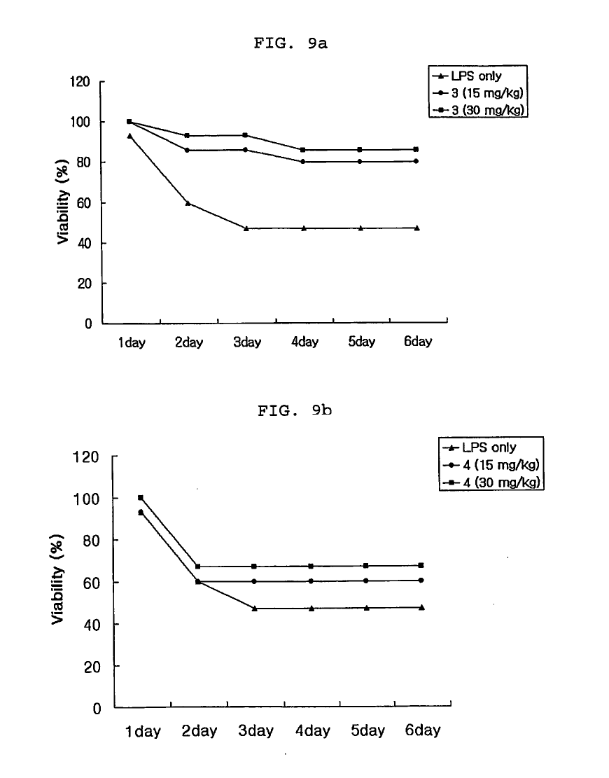

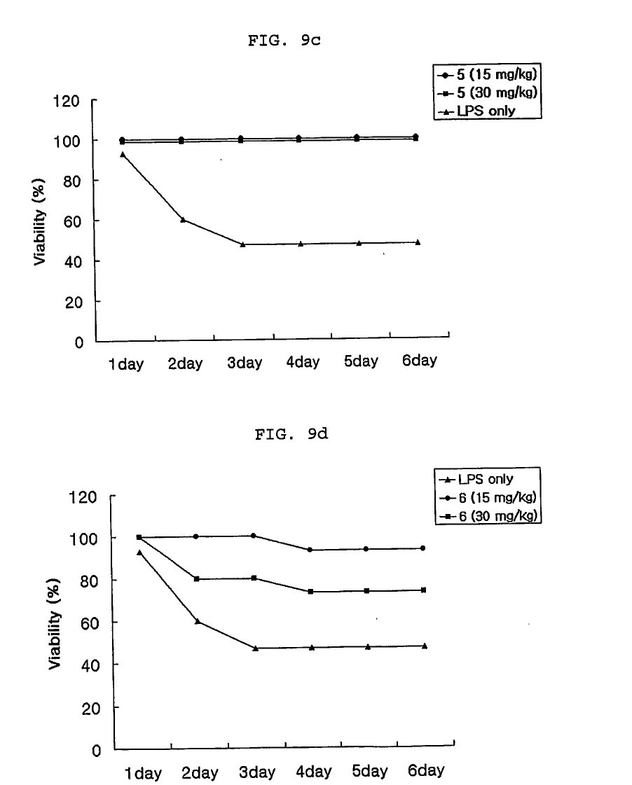

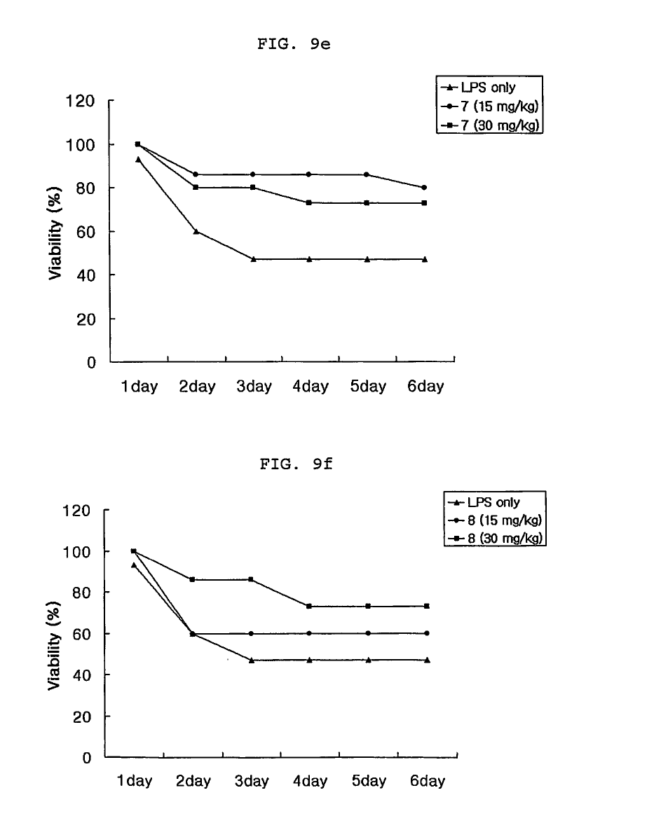

FIG. 9 shows effect of the compound on the survival in LPS-injected rat observed every 24 hours for seven days, where 20 mg/kg of LPS was administered to abdominal cavity of rat, at 30 min after administration of the compound represented by formulas 3 to 8.

FIG. 9a ▴: LPS 20 mg/kg, ●: LPS 20 mg/kg+15 mg/kg of the compound represented by formula 3, ▪: 30 mg/kg of the compound represented by formula 3

FIG. 9b ▴: LPS 20 mg/kg, ●: LPS 20 mg/kg+15 mg/kg of the compound represented by formula 4, ▪: 30 mg/kg of the compound represented by formula 4

FIG. 9c ▴: LPS 20 mg/kg, ●: LPS 20 mg/kg+15 mg/kg of the compound represented by formula 5, ▪: 30 mg/kg of the compound represented by formula 5

FIG. 9d ▴: LPS 20 mg/kg, ●: LPS 20 mg/kg+15 mg/kg of the compound represented by formula 6, ▪: 30 mg/kg of the compound represented by formula 6

FIG. 9f ▴: LPS 20 mg/kg, ●: LPS 20 mg/kg+15 mg/kg of the compound represented by formula 7, ▪: 30 mg/kg of the compound represented by formula 7

FIG. 9g ▴: LPS 20 mg/kg, ●: LPS 20 mg/kg+15 mg/kg of the compound represented by formula 8, ▪: 30 mg/kg of the compound represented by formula 8

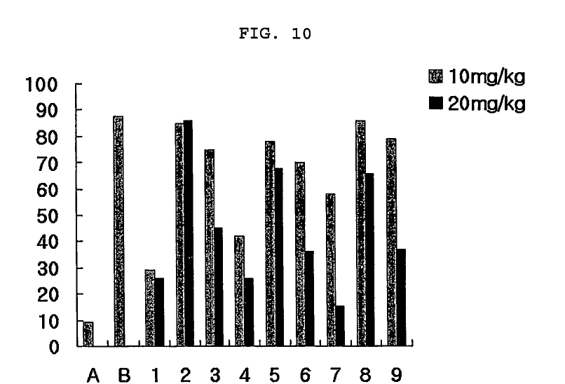

FIG. 10 shows effect of the compound on the serum level of nitrite/nitrate (“NOx”) in LPS-injected group.
A: Normal group
B: LPS(20 mg/kg)
1: LPS(20 mg/kg)+the compound represented by formula 3
2: LPS(20 mg/kg)+the compound represented by formula 4
3: LPS(20 mg/kg)+racemic mixture of the compounds represented by formula 3 and 4
4: LPS(20 mg/kg)+the compound represented by formula 5
5: LPS(20 mg/kg)+the compound represented by formula 6
6: LPS(20 mg/kg)+racemic mixture of the compounds represented by formula 5 and 6
7: LPS(20 mg/kg)+the compound represented by formula 7
8: LPS(20 mg/kg)+the compound represented by formula 8
9: LPS(20 mg/kg)+racemic mixture of the compounds represented by formula 7 and 8

To achieve the above objects, the present invention provides optically active tetrahydroisoquinoline derivatives represented by the following general formula 1 or 2, pharmaceutically acceptable salts thereof and prodrugs thereof:

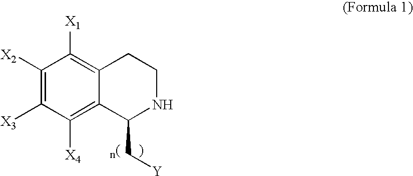

Wherein, X₁, X₂, X₃ and X₄ are independently selected from the group consisting of hydrogen atom, halogen atom, C₁-C₄ alkyl group, hydroxy group and C₁-C₄ alkoxy group; Y represents phenyl group substituted by one or more substituents selected from halogen atom, C₁-C₄ alkyl group, hydroxy group and C₁-C₄ alkoxy group; naphthyl group unsubstituted or substituted by one or more substituents selected from halogen atom, C₁-C₄ alkyl group, hydroxy group and C₁-C₄ alkoxy group; or 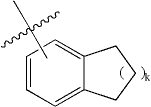
in which, k is an integer of 1-3; and n is an integer of 1 to 3. 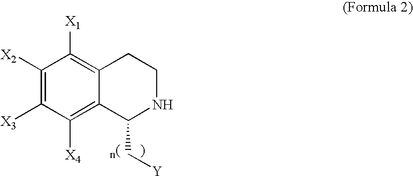
(wherein, X₁, X₂, X₃, X₄, Y and n are as defined in the said formula 1.)

Of the compounds represented by the above general formulas 1 and 2, more preferable are tetrahydroisoquinoline compounds represented by the following formulas 3 to 8:
(S)-6,7-Dihydroxy-1-(p-hydroxyphenylmethyl)-1,2,3,4-tetrahydroisoquinoline, represented by the formula 3;
(R)-6,7-Dihydroxy-1-(p-hydroxyphenylmethyl)-1,2,3,4-tetrahydroisoquinoline, represented by the formula 4;
(S)-6,7-Dihydroxy-1-α-naphthylmethyl)-1,2,3,4-tetrahydroisoquinoline, represented by the formula 5;
(R)-6,7-Dihydroxy-1-(α-naphthylmethyl)-1,2,3,4-tetrahydroisoquinoline, represented by the formula 6;
(S)-6,7-Dihydroxy-1-(β-naphthylmethyl)-1,2,3,4-tetrahydroisoquinoline, represented by the formula 7; and
(R)-6,7-Dihydroxy-1-(β-naphthylmethyl)-1,2,3,4-tetrahydroisoquinoline, represented by the formula 8:

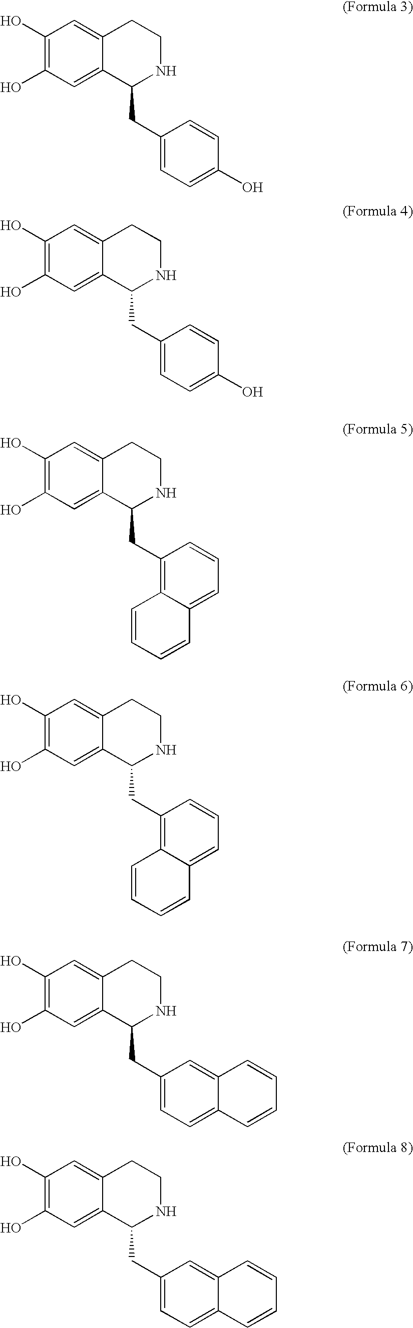

In the present invention, the term “pharmaceutically acceptable salts” means common salts for use in pharmaceutical applications. Examples of the acid used in preparation of the salts include, but are not limited to, hydrochloric acid, bromic acid, sulfuric acid, methansulfonic acid, propionic acid, succinic acid, glutaric acid, citric acid, fumaric acid, maleic acid, tartaric acid, glutamic acid, gluconic acid, glucuronic acid, ascorbic acid, carbonic acid, phosphoric acid, nitric acid, acetic acid, L-aspartic acid, lactic acid, vanilic acid and hydroiodic acid. As well, commercially available acids may be included.

The present invention includes the prodrugs of the compound represented by the general formula 1 or 2. The said prodrugs are functional derivatives of the compound represented by the general formula 1 or 2. Such a prodrug should be easily modifiable to exhibit efficacy of a medicine in vivo. Suitable selection of the prodrug derivatives and general processes for preparation thereof are described in conventional documents (Design of Prodrug, H. Bundgaard 1985).

Further, the present invention provides a method of preparing the compound represented by the general formula 1 or 2. The said method is comprised of the steps:
(1) coupling an acid with an amine, to obtain an amide;
(2) reacting the amide in the presence of POCl₃ (Bischler-Napieralski reaction), to synthesize a cyclic imine salt; thereafter, treating the cyclic imine salt with a base (acid-base reaction), to obtain a corresponding imine;
(3) applying the imine to enantiomerically selective Noyori catalytic reaction, to synthesize an (R) or (S)-precursor, respectively;
(4) treating the said precursor with HBr, to convert one to a salt;
(5) applying the said salt to demethylation reaction; and
(6) removing the halide acid group by neutralizing the said salt, to obtain the compound represented by formula 1 or 2 as free amine.

A method for preparing the compounds represented by formula 3 to 8 as exemplary compounds of tetrahydroisoquinoline derivatives represented by formula 1 or 2 was illustrated in the below.

### (1) Synthesis of (S)-6,7-dihydroxy-1-(p-hydroxyphenylmethyl)-1,2,3,4-tetrahydroisoquinoline (Formula 3)

A method for preparing (S)-6,7-dihydroxy-1-(p-hydroxyphenylmethyl)-1,2,3,4-tetrahydroisoquinoline is comprised of the steps:
condensing p-methoxyphenylacetic acid to 3,4-dimethoxyphenethylamine, to obtain N-(3,4-dimethoxyphenylethyl)(p-methoxyphenyl)acetamide (step 1);
reacting the said compound obtained in the step 1 in the presence of POCl₃ and chloroform, to obtain 6,7-dimethoxy-1-(p-methoxyphenylmethyl)-3,4-dihydroisoquinoline hydrochloride salt (step 2);
reducing the said compound obtained in the step 2 in (R,R)-Noyori catalyst, to (S)-6,7-dimethoxy-1-(p-methoxyphenylmethyl)-1,2,3,4-tetrahydroisoquinoline, thereafter adding acetic acid and halide acid to the obtained compound, to convert corresponding ammonium salt, and neutralizing the said ammonium salt with basic solution, to obtain corresponding free amine thereof (step 3);
adding acetic acid and halide acid to the said compound obtained in the step 3, to obtain corresponding ammonium salt, (S)-6,7-dimethoxy-1-(p-methoxyphenylmethyl)-1,2,3,4-tetrahydroisoquinoline halide acid salt (step 4);
adding BBr₃ to the said compound obtained in the step 4(demethylation reaction), to obtain (S)-6,7-dihydroxy-1-(p-hydroxyphenylmethyl)-1,2,3,4-tetrahydroisoquinoline hydrobromide salt (step 5); and
removing the halide acid salt by neutralizing the said compound obtained in the step 5, to obtain corresponding free amine, (S)-6,7-dihydroxy-1-(p-hydroxyphenylmethyl)-1,2,3,4-tetrahydroisoquinoline (step 6).

### (2) Synthesis of (R)-6,7-dihydroxy-1-(p-hydroxyphenylmethyl)-1,2,3,4-tetrahydroisoquinoline (Formula 4)

(R)-6,7-dihydroxy-1-(p-hydroxyphenylmethyl)-1,2,3,4-tetrahydroisoquinoline was prepared by the same procedure as the method preparing the said S-enantiomer, except using (S,S)-Noyori catalyst in the step 3 instead of (R,R)-Noyori catalyst.

### (3) Synthesis of (S)-6,7-dihydroxy-1-(α-naphthylmethyl)-1,2,3,4-tetrahydroisoquinoline (Formula 5)

A method for preparing (S)-6,7-dihydroxy-1-(α-naphthylmethyl)-1,2,3,4-tetrahydroisoquinoline is comprised of the steps:
condensing α-naphthylacetic acid to 3,4-dimethoxyphenethylamine, to obtain N-(3,4-dimethoxyphenylethyl) (α-naphthyl) acetamide (step 1);
reacting the said compound obtained in the step 1 in the presence of POCl₃ and chloroform, to obtain 6,7-dimethoxy-1-(α-naphthylmethyl)-3,4-dihydroisoquinoline hydrochloride salt (step 2);
reducing the said compound obtained in the step 2 in the presence of (R,R)-Noyori catalyst, to obtain (S)-6,7-dimethoxy-1-(α-naphthylmethyl)-1,2,3,4-tetrahydroisoquinoline, thereafter adding acetic acid and halide acid to the obtained compound, to convert corresponding ammonium salt, and neutralizing the said ammonium salt with basic solution, to obtain corresponding free amine thereof (step 3);
adding acetic acid and halide acid to the said compound obtained in the step 3, to obtain corresponding ammonium salt, (S)-6,7-dimethoxy-1-(α-naphthylmethyl)-1,2,3,4-tetrahydroisoquinoline halide acid salt (step 4);
adding BBr₃ to the said compound obtained in the step 4(demethylation reaction), to obtain (S)-6,7-dihydroxy-1-(α-naphthylmethyl)-1,2,3,4-tetrahydroisoquinoline hydrobromide salt (step 5); and removing the halide acid salt by neutralizing the said compound obtained in the step 5, to obtain corresponding free amine, (S)-6,7-dihydroxy-1-(α-naphthylmethyl)-1,2,3,4-tetrahydroisoquinoline (step 6).

### (4) Synthesis of (R)-6,7-dihydroxy-1-(α-naphthylmethyl)-1,2,3,4-tetrahydroisoquinoline (Formula 6)

(R)-6,7-dihydroxy-1-(α-naphthylmethyl)-1,2,3,4-tetrahydroisoquinoline was prepared by the same procedure as the method preparing the said S-enantiomer, except using (S,S)-Noyori catalyst in the step 3 instead of (R,R)-Noyori catalyst.

### (5) Synthesis of (S)-6,7-dihydroxy-1-(β-naphthylmethyl)-1,2,3,4-tetrahydroisoquinoline (Formula 7)

A method for preparing (S)-6,7-dihydroxy-1-(§-naphthylmethyl)-1,2,3,4-tetrahydroisoquinoline is comprised of the steps:
condensing β-naphthylacetic acid to 3,4-dimethoxyphenethylamine, to obtain N-(3,4-dimethoxyphenylethyl)(β-naphthyl)acetamide (step 1);
reacting the said compound obtained in the step 1 in the presence of POCl₃ and chloroform, to obtain 6,7-dimethoxy-1-(β-naphthylmethyl)-3,4-dihydroisoquinoline hydrochloride salt (step 2);
reducing the said compound obtained in the step 2 in the presence of (R,R)-Noyori catalyst, to obtain (S)-6,7-dimethoxy-1-(β-naphthylmethyl)-1,2,3,4-tetrahydroisoquinoline, thereafter adding acetic acid and halide acid to the obtained compound, to convert corresponding ammonium salt, and neutralizing the said ammonium salt with basic solution, to obtain corresponding free amine thereof (step 3);
adding acetic acid and halide acid to the said compound obtained in the step 3, to obtain corresponding ammonium salt, (S)-6,7-dimethoxy-1-(β-naphthylmethyl)-1,2,3,4-tetrahydroisoquinoline halide acid salt (step 4);
adding BBr₃ to the said compound obtained in the step 4(demethylation reaction), to obtain (S)-6,7-dihydroxy-1-(β-naphthylmethyl)-1,2,3,4-tetrahydroisoquinoline hydrobromide salt (step 5); and
removing the halide acid salt by neutralizing the said compound obtained in the step 5, to obtain corresponding free amine, (S)-6,7-dihydroxy-1-(β-naphthylmethyl)-1,2,3,4-tetrahydroisoquinoline (step 6).

### (6) Synthesis of (R)-6,7-dihydroxy-1-(β-naphthylmethyl)-1,2,3,4-tetrahydroisoquinoline (Formula 8)

(R)-6,7-dihydroxy-1-(β-naphthylmethyl)-1,2,3,4-tetrahydroisoquinoline was prepared by the same procedure as the method preparing the said S-enantiomer, except using (S,S)-Noyori catalyst in the step 3 instead of (R,R)-Noyori catalyst.

Furthermore, the present invention provides a use of optically active compound represented by formula 1 or 2, as a therapeutic agent of septicemia, heart failure, hypertension, thrombosis, inflammation, disseminated intravascular coagulation (DIC) and cardiac insufficiency.

The present inventors disclosed that racemic compounds represented by formula 1 or 2 had a use thereof as therapeutic agent of septicemia, heart failure, hypertension, thrombosis, inflammation, disseminated intravascular coagulation (DIC) and cardiac insufficiency (KR patent No. 352425, PCT/KR99/00631). Therefore, we intended to describe the said use of optical isomer thereof.

Furthermore, the present invention provides a pharmaceutical composition comprising a compound selected from the compound represented by formula 1 or 2, preferably the compound represented by formula 3 to 8 as an effective ingredient. The said composition can be used for a therapeutic agent of cardiac insufficiency, heart failure, hypertension, thrombosis, inflammation, septicemia or DIC.

More specifically, the above composition is used for prophylaxis or treatment of heart failures caused by decrease of myocardial contractile force due to congestive heart failure; ischemic heart diseases; iNOS increase in chronic inflammation; and circulatory disorders by hypertension, arteriosclerosis and coronary artery diseases; thrombogenesis in ischemic cerebral vascular disorder, coronary artery disease, ischemic myocardial infarction, chronic arterial obstruction, thrombosis or embolism after surgery, induced by thrombus; damages by ischemia and reperfusion including inflammatory diseases, arteriosclerosis, myocardial infarction, cerebral apoplexy due to damage of tissues and organs; septicemia caused by damage of multiple organs and disseminated intravascular coagulation; and disseminated intravascular coagulopathy caused by drastic decrease of platelet number, bleeding, shock, thrombus, and vascular obstruction due to activation of rapid blood coagulation.

To treat cardiac insufficiency, drug selected from cardenoides, vasodilatin, calcium antagonist, angiotensin converting enzyme (ACE) inhibitor was adiministered as sole or combination treatment in order to improve decreased myocardial contractile force and relieve the heart. This theraphy, combined-treating this drug to improve circulatory activity and induce cordial activity has so-called hemodynamic favorable-turn. The compound represented by formula 1 or 2 is inodilator, material that has cordial activity and vasodilation simulataneously, and fulfills the said two requirements.

Also, cardiac insufficiency intends to be serious if vascular obstruction such as arteriosclerosis or hypertension is lasted for a long time, and this imposes a burden on heart. Also, cardiac insufficiency intends to be serious by coronary artery disease caused by thrombosis, or ischemic heart disease such as myocardial infarction. Thus, inhibitor of platelet aggregation must be lastly administered to the patient easily taken to the heart disease, for the prevention of relapse or advance of heart disease or vascular obstruction. Therefore, the compound represented formula 1 or 2 has anti-thrombotic activity by inhibiting platelet aggregation, and fulfills another requirement as a therapeutic agent of cardial insufficiency. Therefore, the compound represented by formula 1 or 2 of the present invention is a surprising therapeutic agent of cardiac insufficiency, for the compound exhibits the said pharmaceutical workings both simultaneously and complexly

The said therapeutic agent functions to treat cardiac insufficiency or inhibit a progress thereof caused by decrease of myocardial contractile force due to acute myocardial infarcation, immunity increase such as chronic inflammation, ischemic heart disease, congestive heart-failure such as protracted hypertension, arteriosclerosis, coronary artery disease.

Furthermore, the pharmaceutical composition comprising the compound represented by formula 1 or 2 can be used as thereapeutic agent for thrombosis, tissue injury, septicemia or disseminated intravascular coagulation.

The said therapeutic agent of thrombosis can treat or prevent ischemic cerebral vascular disorder, coronary artery disease, ischemic myocardial infarction or chronic arterial obstruction caused by thrombosis. Further, the said agent can prevent or inhibit formation of thrombosis in operation or embolus.

Also, the said therapeutic agent of tissue injury can treat inflammatory disease caused by tissue or organ injury. Further, the said agent can treat or prevent another tissue injury caused by ischemia and reperfusion containing arthritis, arteriosclerosis, myocardial infarction or cerebral apoplexy. Further, the said agent can inhibit a progress thereof.

Also, the said therapeutic agent of disseminated intravascular coagulopathy can treat symptoms caused by drastically decreased of platelet number, bleeding, shock, thrombus, vascular obstruction due to activation of blood coagulation.

Also, the said therapeutic agent of septicemia in accordance to the present invention can treat septicemia induced by disseminated intravascular coagulation and multiple organ failure.

In experiment investigating effect of enantiomer of the present invention as the compounds represented by formulas 1 or 2, effect of the compound represented by formula 1 (S-enantiomer) was more excellent than one of the compound represented by formula 2(R-enantiomer), where the effect was excellent relative to one of the racemic mixture.

In experiment for investigating an effect of contractile force and heart rates of auricle muscles excised form rats, R-enantiomer represented by the formula 4 increased the contractile force and heart rates of auricle muscles, however the effect of the said R-enantiomer was less than one of S-enantiomer represented by the formula 3. R-enantiomer represented by the formula 6 or 8 have no effect to the contractile force and heart rates of auricle muscles, however, S-enantiomer represented by the formula 5 or 7 strongly increased contractile force of the auricle muscles and thus strongly stimulated their beating. Also, the racemic mixture increased the contractile force and heart rates of auricle muscles, weakly than S-enantiomer did, or strongly than R-enantiomer did (shown in table 1a and 1b).

In experiment for investigating an effect of the compounds on thoracic aortas, all of the compounds represented by the formulas 3 to 8 induced atony of contracted blood vessels in a concentration-dependant manner. In addition, all of the S-enantiomers (formulas 3, 5 and 7) showed a stronger vasodilation effect than the R-enantiomers (formulas 4, 6 and 8). However, the racemic mixture showed vasodilation effect, weaker than S-enetiomer did, or stronger than R-enantiomer did. (shown in table 2)

In experiment for investigating an effect of the compounds on nitrite synthesis in macrophage cell stimulated by LPS and IFN-r, the compounds of the present invention inhibited production of NO, in inverse proportion to added concentration of the compounds. For example, NO produced in group treated with only LPS/IFN-r was 37.2 μM. However, when the compound represented by the formula 3, S-enantiomer was treated in the said macrophage cell at various concentrations of 10, 50, and 100 μM, production of NO was reduced to 30.5, 22.3, and 18.2 μM, respectively, in inverse proportion to added concentration of the compounds. Also, the compound represented by formula 4, R-enantiomer inhibited production of NO weakly than S-enantiomer did. Also, the racemic mixture of the compound represented by the formula 4 inhibited production of NO, weakly than S-enantiomer did or strongly than R-enantiomer did. And another S-enantiomer, the compound represented by formula 5 and 7 inhibited production of NO, in inverse proportion to added concentration of the compounds. However, another R-enantiomer, the compound represented by formula 6 and 8 never or slightly inhibited production of NO. The racemic mixture of the corresponding compound inhibited production of NO, weakly than S-enantiomer did or strongly than R-enantiomer did. (showin in table 3)

In experiment for investigating an effect of the compound on expression of iNOS stimulated by LPS, expression of iNOS mRNA was decreased by the compound of the present invention. Particularly, S-enantiomer, the compounds represented by formula 3, 5 or 7 inhibited expression of iNOS mRNA strongly than R-enantiomer, the compounds represented by formulas 4, 6 or 8. Also, S-enantiomer inhibited expression of iNOS mRNA much strongly than the racemic mixture did. (shown in FIG. 1)

On platelet aggregation induced by AA, all of the compounds of the present invention showed much stronger than inhibitory effect than that on platelet aggregation induced by ADP or collagen. Particularly, S-enantiomer had inhibitory effect of platelet aggregation about two times as strong as R-enantiomer did.

On platelet aggregation induced by epinephrine, all of the compounds of the present invention showed much stronger than inhibitory effect than that on platelet aggregation induced by ADP, collagen, AA or U46619. S-enantiomer had inhibitory effect of platelet aggregation about 6˜17 times as strong as R-enantiomer did. In addition, S-enantiomer, the compounds represented by formulas 3, 5 or 7 had inhibitory effect of platelet aggregation about five times as strong as the racemic mixture did. (shown in FIG. 4)

To investigate effect of enantiomer on the various variables in rats where disseminated intravascular coagulation and multiple organ failure was induced by endotoxin, LPS was injected to the vein of rat for 4 hours. In the experiment, platelet was decreased, (shown in FIG. 2), fibrinogen concentration was low (shown in FIG. 3), the serum level of FDP was increased (shown in FIG. 4), PT or APTT time was increased (shown in FIG. 5 or 6), the serum level of AST or BUN was increased (shown in FIG. 7 or 8). Also, all the compounds of the present invention have inhibitory effect on decrease of platelet or concentration of fibrinogen by LPS. Also, all the compounds of the present invention have inhibitory effect on decrease of the serum level of FDP, increase of PT or APTT time or increase of the serum level of AST or BUN. Also, S-enantiomer, the compounds represented by formulas 3, 5 or 7 has the inhibitory effect, where the effect is stronger than one of R-enantiomer, the compounds represented by formulas 4, 6 or 8. (shown in FIG. 2˜8)

To investigate effect of the compound on the survival in LPS-injected group, LPS was administered to the abdominal cavity of the rat in an amount of 20 mg/kg, where the dosage is quantity that half of rats was deceased. Thereafter, the compound of the present invention was administered, survival was observed for six days. As shown in FIG. 9, the survival of the rat to which all the compounds represented by formulas 3 to 8 was higher than one of the rat to which LPS only was administered. In addition, the survival of the rat to which S-enantiomer, the compound represented by formulas 3, 5 or 7 was administered was higher than one of the rat to which R-enantiomer, the compound represented by formulas 4, 6 or 8 was administered. The survival of the rat to which S-enantiomer, the compound represented by formula 3, 5 or 7 was administered in an amount of 15 mg/kg, after six days was 80%, 100% or 73%, respectively. However, the survival of the rat to which LPS only was injected was much lower than one of the rat to which all the compounds was administered, respectively. When the compound represented by formula 3 or 5 was administered in an amount of 30 mg/kg than 15 mg/kg, the survival increased. However, when the compound represented by formula 7 was administered in the same amount, the survival decreased by 7%. Also, the survival of the rat to which R-enantiomer, the compound represented by formula 4, 6 or 8 was administered in an amount of 15 mg/kg, after six days was 60%, 73% or 90%, respectively. Also, the survival of the rat to which R-enantiomer, the compound represented by formula 4, 6 or 8 was administered in an amount of 30 mg/kg, after six days was 67%, 93% or 72%, respectively, where the survival is higher than one of the rat to which the compound was administered in an amount of 15 mg/kg. Therefore, S-enantiomer, the compound represented by formula 3, 5 or 7 showed the survival higher than R-enantiomer, the compound represented by formula 4, 6 or 8. When dosage increased from 15 mg/kg to 30 mg/kg, all the compounds, except the compounds represented by formula 7 increased the survival. When the compounds represented by formula 5, of S-enantiomer was administered to the rat in an amount of 15 mg/kg or 30 mg/kg, all the rats were not diseased (survival: 100%). Therefore, the compound represented by formula 5 has most excellent effect of all the compounds. However, the survival of the rat to which higenamine (the racemic mixture of the compounds represented by formula 3 and 4), or the racemic mixture of the compounds represented by formula 5 and 6 was administered was 80% or 90%, respectively. (Kang et al., J. Pharmacol. Exp. Ther., 1999, 291, 314-320) (Kang et al., J. Pharmacol. Exp. Ther., 1999, 128, 357-364)

Also, in the experiment investigating that whether the compound has inhibitory effect on the serum level of nitrite/nitrate in LPS-injected group, the plasma level of NOₓ derived from the group to which the compound of the present invention was administered was reduced. Also, S-enantiomer has inhibitory effect on the plasma level of NOₓ, where the effect is stronger than one of R-enantiomer (shown in FIG. 10).

Pharmaceutical composition of the present invention can be administered in various methods such as oral administration, parenteral administration, or rectal administration. The composition can be formulated in the various formulation such as injection, capsule, dragee, grain, solution, suspension, emulsion or auxiliary. The composition contains pharmaceutically acceptable carrier such as organic or inorganic material, solid, semi-solid, liquid or diluting agent. Also, if necessary, additive agent conservatively used, for example, adjuvant, stabilizing agent, wetting agent, emulsifying agent, buffering solution or other commonly used additives was contained in the said pharmaceutical composition.

Concentration of the compound of the present invention was maintained steadily in oral administration for a long time. Therefore, in injection or oral administration, the dosage of the compound is 0.01-5 mg/kg or 2-200 mg/kg, respectively. However, the dosage of the compound represented by formula 1 or 2 was decided, depending upon age, body weight, sex and state of the patients, seriousness of disease under treatment. Also, the compound was regularly administered one or more times per day, depending upon the prescription of doctor or pharmacist.

A better understanding of the present invention may be obtained in light of the following preparation examples, examples and test examples which are set forth to illustrate, but are not to be construed to limit the present invention.

### PREPARATION EXAMPLE 1

### Synthesis of Di-μ-chloro-bis[(η⁶-p-cymene)chlororuthenium (II)

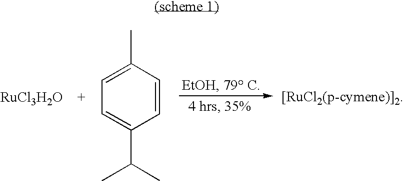

In a flask, RuCl₃H₂O (514 mg, 1.97 mmol) was dissolved in ethanol (25 ml), to which α-phellandrene (3.51 mL, 21.6 mmol) was added dropwise. Then, the flask, equipped with a reflux condenser, was filled with nitrogen gas. Using a thermostat, the reaction solution was adjusted to 79° C. in temperature, and then refluxed for 4 hours. The reaction mixture was cooled to room temperature. After that, the precipitated solid was filtered through a Buchner funnel. Thus obtained brown solid was washed once with methanol (40 ml), followed by removing the solvent under reduced pressure. The brown solid (340 mg) was recrystallized from methanol (3 mL), to give the desired compound, Di-μ-chloro-bis[(η⁶-p-cymene)chlororuthenium (II) (211 mg, 35%) as a brown solid.

¹H-NMR(300 MHz, DMSO-d₆): ε5.77(q,4H), 2.8(m,1H), 2.1(s,3H), 1.2(d,6H)<

### PREPARATION EXAMPLE 2

### Synthesis of RuCl[TsDPEN](p-cymene) Catalyst

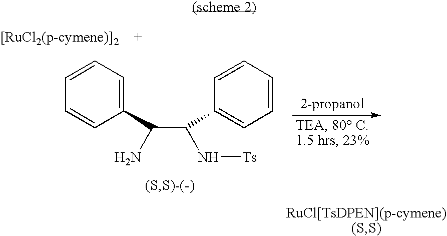

In a flask, Di-μ-chloro-bis[(η⁶-p_cymene)chlororuthenium (II) (211 mg, 345 μmol) was dissolved in 2-propanol (10 mL), to which triethylamine (TEA) (0.192 mL, 1.38 mmol) was added and then (1S,2S)-(p-toluenesulfonyl)-1,2-diphenylethylenediamine (253 mg, 689 μmol) was added dropwise. Then, the flask, equipped with a reflux condenser, was filled with nitrogen gas. Using a thermostat, the reaction solution was adjusted to 80° C. in temperature and refluxed for 1.5 hours. Completion of the reaction was detected by thin film chromatography. The reaction mixture was cooled to room temperature, and vacuum concentrated, to give a very viscous liquid residue. Such residue was dissolved in methanol (1 ml) with mild heating, and allowed to stand for one day and night. A scarlet solid was precipitated, and only deep brown supernatant was discarded. The residual precipitate as a scarlet solid was washed once with ethanol (1 mL). The solvent was removed under reduced pressure, yielding the desired compound, RuCl[TsDPEN](p-cymene) (100 mg, 23%) as a scarlet solid.

¹H-NMR(300 MHz, CDCl₃): δ 6.4-7.1(m,14H), 5.69-5.73(m,4H), 3.7(d,1H), 3.56(d,1H), 3.1(m,1H), 2.4(s,3H), 2.2(s,3H), 1.39-1.40(d,6H)<

### EXAMPLE 1

### Synthesis of (R)-6,7-dihydroxy-1-(p-hydroxyphenylmethyl)-1,2,3,4-tetrahydroisoquinoline hydrobromide salt

### (step 1):Synthesis of N-(3,4-Dimethoxyphenethyl)(p-methoxyphenyl)acetamide

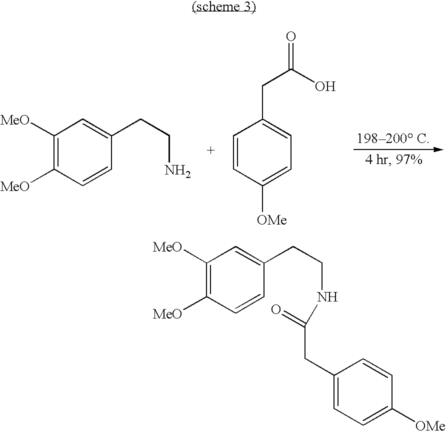

To p-methoxyphenyl acetic acid (50.4 g, 0.303 mol) in a 50 ml round-bottom flask, 3,4-dimethoxyphenethylamine (51.2 mL, 0.303 mol) was added dropwise. Then, the flask, equipped with a septum, was charged with nitrogen gas. Using the thermostat, the reaction solution was adjusted to the temperature of 198-200° C. and heated for 4 hours. Completion of the reaction was detected by thin film chromatography. The reaction mixture was cooled to the room temperature, and chloroform (500 mL) was added thereto to dissolve the produced precipitate. The chloroform solution was successively washed with 1 N HCl, 1 N NaHCO₃ and saturated brine, dried over anhydrous magnesium sulfate and filtered with a glass filter. The filtrate was vacuum concentrated to give a yellowish solid, which was then dissolved in a minimal amount of chloroform, added with ether (500 mL) and stirred, to obtain a white solid. Such solid was filtered through a Buchner funnel, followed by removing the solvent under reduced pressure, to yield the desired compound, N-(3,4-Dimethoxyphenethyl)(p-methoxyphenyl)acetamide (96.8 g, 97%) as a white solid.

m.p=117° C.

R_{f}: 0.38 (hexane:ethyl acetate=0.5:1)

¹H-NMR(300 MHz, CDCl₃) δ 6.5-7.1(m,7H), 5.4(br,1H), 3.80(s,3H), 3.81(s,3H), 3.85(s,1H), 3.46(s,2H), 3.4(t,2H), 2.6(t,2H)

IR(KBr pellet, cm⁻): 3322, 3002, 2942, 2845, 1653, 1521, 4170

HRMs: m/z calcd for C₁₉H₂₃NO₄ (M+): 329.16. Found: 329.01

Anal. calcd for C₁₉H₂₃NO₄: C,69.28; H,7.04; N,4.25. Found: C,69.26; H,7.12; N,4.27.

### (Step 2): Synthesis of 6,7-Dimethoxy-1-(p-methoxyphenylmethyl)-3,4-dihydroisoquinoline hydrochloride salt

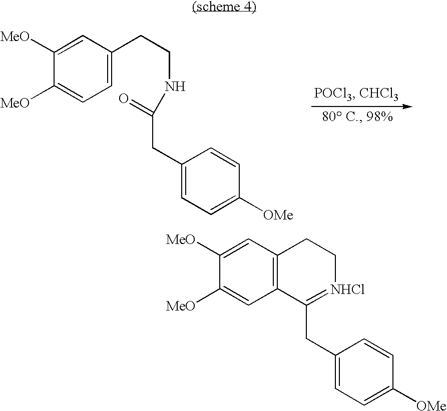

In a 500 ml round-bottom flask, N-(3,4-Dimethoxyphenethyl)(p-methoxyphenyl)acetamide (96 g, 0.291 mol) was dissolved in chloroform (600 mL). POCl₃ (109 mL, 1.17 mol) was added dropwise thereto, and the flask, equipped with a reflux condenser, was filled with nitrogen gas. Using the thermostat, the reaction solution was adjusted to the temperature of 80° C. and refluxed for 33 hours. Completion of the reaction was detected by thin film chromatography. The chloroform solvent was removed under reduced pressure. The produced light green solid was dissolved in a minimal amount of chloroform, to which distilled ethyl acetate (400 mL) was added, with stirring, to precipitate a solid. Such a solid was filtered via a Buchner funnel, followed by removing the solvent under reduced pressure, to give the desired compound, iminium salt (99.2 g, 98%) as a light-green solid.

m.p=120° C.

R_{f}: 0.38 (hexane:ethyl acetate=0.5:1)

¹H-NMR(300 Hz,DMSO): 7.57(s,1H), 7.39(d,2H), 7.12(s,1H), 6.92(d,2H), 4.41(s,2H), 3.82(s,3H), 3.79(s,3H), 3.64(s,3H), 3.01(t,2H), 2.43(s,2H)

HRMs: m/z calcd for C₁₉H₁₂NO₃ (M+): 347.13. Found: 353.55

### (Step 3): Synthesis of (R)-6,7-dimethoxy-1-(p-methoxyphenylmethyl)-1,2,3,4-tetrahydroisoquinoline

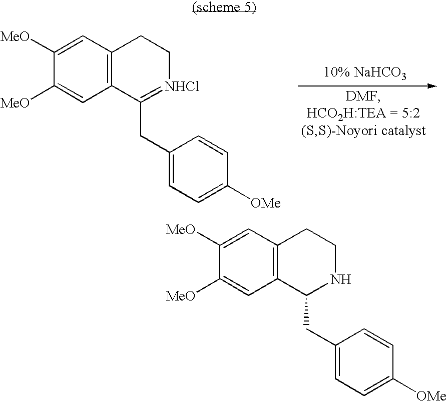

The iminium salt (30.2 g, 0.087 mol) was dissolved in chloroform (200 mL) and 10% aqueous NaHCO₃ solution (100 mL) was slowly added thereto dropwise at 0° C. the reaction mixture was stirred at 0° C. for 1 hour. The reaction mixture was extracted with chloroform (3×80 mL), and the combined organic layer was dried over anhydrous magnesium sulfate and filtered with the glass filter. The filtrate was concentrated under reduced pressure, to give 6,7-dimethoxy-1-(4-methoxyphenylmethyl)-3,4-dihydroisoquinoline (23 g, 85%) as a pale yellow solid. Obtained imine was dissolved in DMF (250 mL), to which (S,S)—Ru (II) catalyst (0.47 g, 0.74 mmol) was added and then distilled HCO₂H:TEA=5:2 was added thereto dropwise. The flask, charged with nitrogen gas, was equipped with a septum. Then, the reaction mixture was stirred for 12 hours. While completion of the reaction was detected by TLC, the reaction was terminated with 20% aqueous Na₂CO₃ solution. As such, 20% aqueous Na₂CO₃ solution was used only to the extent of obtaining a slightly basic reaction mixture. Such a mixture was extracted with methylene chloride, and the organic layer was washed with saturated brine, dried over anhydrous magnesium sulfate and filtered via the glass filter. Thereafter, the filtrate was concentrated under reduced pressure to give a crude product as a deep green liquid, which was then purified by chromatography, yielding a corresponding amine.

The said amine was dissolved in acetic acid (60 mL), to which 48% HBr (22 mL) was added dropwise. The said mixture was stirred for 2 hours to give a deep green precipitation.

Ether (250 ml) was added to the said precipitation to give a emulsion. The said emulsion was stirred for I hour, thereafter the supernatant was discarded. Ether (300 ml) was added to the emulsion, stirred for 1 hour. The solid was filtered and washed with ethyl acetate. Then, solvent was removed under reduced pressure to give an ammonium salt as a light green solid. The said ammonium salt was dissolved in the solution prepared by mixing dichloromethane and methanol in a ratio of 5:1. n-hexane was added to the solution, to recrystallize a crystal (17.9 g). The crystal was dissolved in chloroform (90 ml), thereto 2N NaOH (70 ml) was added at 0° C. The aqueous layer was extracted with chloroform three times. The organic layer was washed with saturated brine, dried over MgSO₄, filtered and concentrated under reduced pressure to give amine. The said purification process was repeated one more times, to give a white solid free amine (14.3 g, 62%). Purity (98% or more) and ee value (99% or more) of the product was measured by use of HPLC (Daicel Chiralcel OD 4.6 mm×25 cm column: developing liquid-hexane:2-propanol:diethylamine=40:10:0.05; flowing rate—0.5 mL/min; retention time—46.5 min).

m.p=195° C.

R_{f}: 0.32 (ethanol:hexane=3:1)

¹H-NMR(300 MHz,CDCl₃) δ 7.2(d,2H), 6.8(d,2H), 6.6(s,2H), 6.0(s,1H), 5.18(s,1H), 4.7(s,1H), 3.2-3.0(m,3H)

HRMs: m/z calcd for C₁₉H₂₃NO₂ (M+): 313.39. Found: 313.55

[α]²⁸_{D}=+25.0 (c=0.05, CHCl₃)

### (Step 4): Synthesis of (R)-6,7-Dimethoxy-1-(p-methoxyphenylmethyl)-1,2,3,4-tetrahydroisoquinoline hydrobromide salt

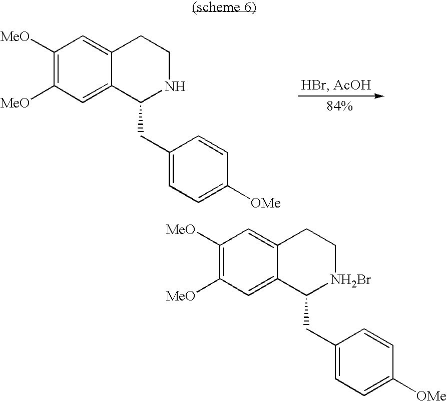

In a 1 L round-bottom flask, the said amine (14.3 g, 45.6 mmol) was dissolved in acetic acid (50 ml). To the above solution, 48% HBr (18 mL) was added dropwise. Within 1 minute, ammonium salt was produced, to which ether (100 mL) was then added. The solution was stirred for 1 hour, washed with ethyl acetate two times and filtered via a Buchner funnel. The solvent was removed under reduced pressure, to yield the desired compound, ammonium salt (15 g, 70%) as a white solid. The said ammonium salt was dissolved in a solution prepared by mixing methanol and methylene chloride in a ratio of 1:5, thereto hexane was added. The solid was grinded to give pure ammonium salt (15.1 g, 84%)

m.p=195° C.

¹H-NMR(300 MHz, DMSO-d₆) δ 7.4(d,2H), 7.04(d,2H), 6.88(s,1H) 4.72(s, 1H), 6.6(s,1H), 4.72(s,1H) 3.85(d,6H), 3.61(s,3H), 3.5-3.0(m)

HRMs: m/z calcd for C₁₉H₂₄BrNO₃ (M+):393.09. Found: 391.58

Anal. calcd for C₁₉H₂₄BrNO₃: C,57.88; H,6.14; N,3.55. Found: C,57.88; H,6.19; N,3.58.

IR(KBr pellet, cm⁻¹): 2945, 2780, 2650, 2453, 1618, 1582, 1516

### [α]²⁸_{D}=−25.0 (c=0.05, MeOH)

### (Step 5) Synthesis of (R)-6,7-dihydroxy-1-(p-hydroxyphenylmethyl)-1,2,3,4-tetrahydroisoquinoline hydrobromide salt

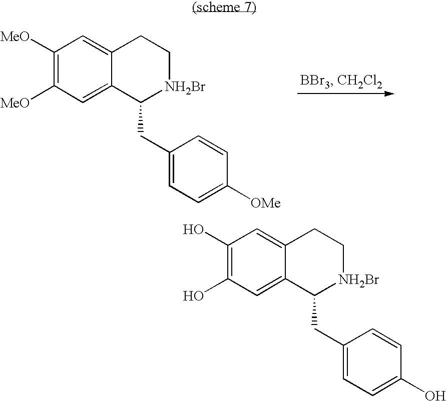

The said ammonium salt (15.1 g, 38.4 mmol) in a 50 mL round-bottom flask was dissolved in methylene chloride (150 mL). The flask was filled with nitrogen gas, to which BBr₃ (77 mL) was slowly added dropwise at −78° C. The reaction temperature was gradually increased to 0° C. and the reaction mixture was stirred for 3 hours. While completion of the reaction was detected by NMR, the reaction was terminated with H₂O and ethanol. The reaction mixture was stirred for 1 hour, and the produced precipitate was washed with ethyl acetate two times, and filtered via a Buchner funnel. The solvent was removed under reduced pressure, to give (R)-6,7-dihydroxy-1-(4-hydroxyphenylmethyl)tetrahydroisoquinoline hydrobromide salt as a white solid (10.5 g, 78%). Purity (98% or more) and ee value (99% or more) of the product were measured by use of HPLC (CHIREX 3020G-EO, Phenomenex Co., U.S.A., 4.6 mm×25 cm column: developing solvent—hexane: methylene chloride:trifluoroacetic acid/ethanol (1/20)=53:35:12; flowing rate—0.9 mL/min; retention time—19.6 min).

m.p=249° C.

R_{f}: 0.50 (3 drops of 28% aqueous ammonium hydroxide solution were added to a mixed developing-solvent of benzene:acetone:MeOH=5:4:2)

¹H-NMR(300 MHz, DMSO-d₆) δ 9.35(br,1H), 9.06(br, 1H), 8.84(br,1H), 7.1(d,2H), 6.7(d,2H), 6.5(d,2H), 2.79-3.17(m,6H)

HRMs: m/z calcd for C₁₆H₁₈BrNO₃ (M+):351.05. Found: 350.61

Anal. calcd for C₁₆H₁₈BrNO₃: C,54.56; H,5.15; N,3.98. Found: C,54.55; H,5.12; N,4.02.

IR(KBr pellet, cm⁻¹): 3231, 2798, 1627, 1520, 1454

[α]²⁸_{D}=+25.0 (c=0.05, MeOH)<

### EXAMPLE 2

### Synthesis of (S)-6,7-dihydroxy-1-(p-hydroxyphenylmethyl)-1,2,3,4-tetrahydroisoquinoline hydrobromide salt

Step 1 and step 2 were accomplished in the same procedure as ones of the said example 1.

### (Step 3): Synthesis of (S)-6,7-dimethoxy-1-(p-methoxyphenylmethyl)-1,2,3,4-tetrahydroisoquinoline

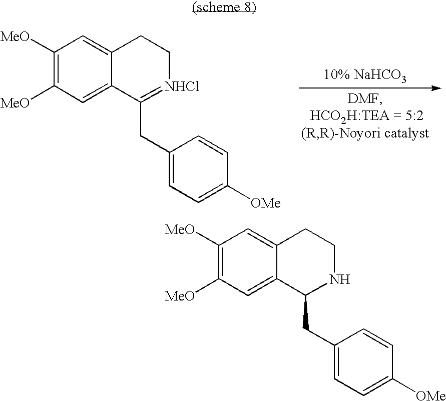

The iminium salt (33.5 g, 96 mmol) was dissolved in chloroform (200 mL), to which 10% aqueous NaHCO₃ solution (100 mL) was slowly added dropwise at 0° C., and stirred at 0° C. for 1 hour. The reaction mixture was extracted with chloroform (3×80 mL), and the combined organic layer was dried over anhydrous magnesium sulfate and filtered with the glass filter. The filtrate was concentrated under reduced pressure, to give 6,7-dimethoxy-1-(4-methoxyphenylmethyl)-3,4-dihydroisoquinoline (29 g, 96%) as a pale yellow solid. The obtained imine was dissolved in DMF (250 mL), to which (R,R)—Ru (II) catalyst (0.601 g, 0.93 mmol) was added and then distilled HCO₂H:TEA=5:2 (53 ml) were added dropwise. The flask, filled with nitrogen gas, was equipped with a septum. Then, the reaction mixture was stirred for 12 hours. While completion of the reaction was detected by TLC, the reaction was terminated with 20% aqueous Na₂CO₃ solution. As such, 20% aqueous Na₂CO₃ solution was used only to the extent of obtaining a slightly basic reaction mixture. Such a mixture was extracted with methylene chloride, and the organic layer was washed with saturated brine, dried over anhydrous magnesium sulfate and filtered via the glass filter. The filtrate was concentrated under reduced pressure to give a crude product as a deep green liquid.

The said amine was dissolved in acetic acid (60 mL) and 48% HBr (22 mL) was added thereto dropwise. The said mixture was stirred for 2 hours to give a deep green precipitate. Ether (250 mL) was added to the said precipitate to give a solution. The solution was stirred for 1 hour and the supernatant was discarded, followed by filtering the stirred solution via a Buchner funnel. Ether (300 ml) was added to the precipitate to give an emulsion, the said emulsion was stirred for 1 hour, filtered to give a solid product. The said solid product was washed with ethyl acetate. Then, the solvent was removed under reduced pressure, to give an ammonium salt as a light green solid. The said ammonium salt was dissolved in solution prepared by mixing dichloromethane and methanol in a ratio of 5:1. n-hexane was added to the solution to recrystallize a crystal (20.1 g). The crystal was dissolved in chloroform (90 ml), therein 2N NaOH (70 ml) was added at 0° C. The aqueous layer was extracted with chloroform three times. The organic layer was washed with saturate brine, dried over MgSO₄, filtered and concentrated under reduced pressure to give amine (16.6 g). The said purification process was repeated one more times, to give a white solid free amine (16.2 g, 62%). Purity (98% or more) and ee value (99% or more) of the product was measured by use of HPLC (Daicel Chiralcel OD 4.6 mm×25 cm column: developing liquid-hexane:2-propanol:diethylamine=40:10:0.05; flowing rate—0.5 mL/min; retention time—25.4 min).

m.p.: 195° C.

R_{f}: 0.32 (ethanol:hexane=3:1)

¹H-NMR(300 MHz, CDCl₃) δ 7.2(d,2H), 6.8(d,2H), 6.6(s,2H), 6.0(s,1H), 5.18(s,1H), 4.7(s,1H), 3.2-3.0(m,3H)

HRMs: m/z calcd for C₁₉H₂₃NO₂ (M+): 313.39. found 313.55

### (Step 4): Synthesis of (S)-6,7-dimethoxy-1-(p-methoxyphenylmethyl)-1,2,3,4-tetrahydroisoquinoline hydrobromide salt

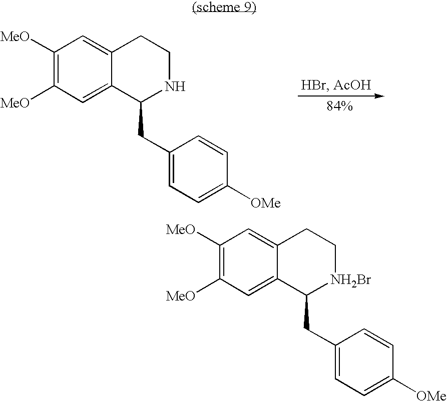

In a 1 L round-bottom flask, the said amine (16.1 g, 0.046 mol) was dissolved in acetic acid (46 mL). To the above solution, 48% HBr (15 mL) was added dropwise. Within 1 minute, ammonium salt was produced, to which ether (150 mL) was added, stirred for 1 hour, washed with ethyl acetate two times, and filtered via a Buchner funnel. The solvent was removed under reduced pressure, to yield the desired compound, ammonium salt as a white solid. The said ammonium salt was dissolved in the solution prepared by mixing methanol and methylene chloride in a ratio of 1:5. Hexane was added to the resultant solution, grinded to give pure ammonium salt (17.1 g, 84%)

m.p=195° C.

¹H-NMR(300 MHz, DMSO-d₆) δ 7.4(d,2H), 7.04(d,2H), 6.88(s,1H) 4.72(s,1H), 6.6(s,1H), 4.72(s,1H) 3.85(d,6H), 3.61(s,3H), 3.5-3.0(m)

HRMs: m/z calcd for C₁₉H₂₄BrNO₃ (M+):393.09. Found: 391.38

Anal. calcd for C₁₉H₂₄BrNO₃: C,57.88; H,6.14; N,3.55. Found: C,57.89; H,6.19; N,3.57.

IR(KBr pellet, cm⁻¹): 2880, 2766, 2555, 1620, 1580, 1509

[α]²⁸_{D}=+25.6 (c=0.052, MeOH)

### (Step 5): Synthesis of (S)-6,7-dihydroxy-1-(p-hydroxyphenylmethyl)-1,2,3,4-tetrahydroisoguinoline hydrobromide salt

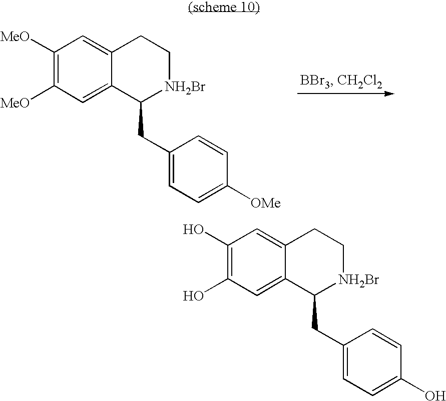

In a 500 mL round-bottom flask, the ammonium salt (17.1 g, 43.6 mmol) was dissolved in methylene chloride (300 mL). The flask was filled with nitrogen gas, to which BBr₃ (87 mL) was slowly added dropwise at −78° C. The reaction temperature was gradually increased to 0° C. and the reaction mixture was stirred for 3 hours. While completion of the reaction was detected by NMR, the reaction was terminated with H₂O and ethanol. The reaction mixture was stirred for 1 hour, washed with ethyl acetate two times, and the produced precipitate was filtered via a Buchner funnel. The solvent was removed under reduced pressure, to give (S)-6,7-dihydroxy-1-(4-hydroxyphenylmethyl)tetrahydroisoquinoline hydrobromide salt as a white solid (11.6 g, 76%). Purity (98% or more) and ee value (99% or more) of the product were measured by use of HPLC (CHIREX 3020G-EO, Phenomenex Co., U.S.A., 4.6 mm×25 cm column: developing solvent-hexane:methylene chloride:trifluoroacetic acid/ethanol (1/20)=53:35:12; flowing rate—0.9 mL/min; retention time-26.6 min).

m.p=249° C.

R_{f}: 0.50 (3 drops of 28% aqueous ammonium hydroxide solution were added to a mixed developing-solvent of benzene:acetone:MeOH=5:4:2)

¹H-NMR(300 MHz, DMSO-d₆) δ 9.35(br,1H), 9.06(br,1H), 8.84(br,1H), 7.1(d,2H), 6.7(d,2H), 6.5(d,2H), 2.79-3.17(m,6H)

HRMs: m/z calcd for C₁₆H₁₈BrNO₃ (M+):351.05. Found: 350.58

Anal. calcd for C₁₆H₁₈BrNO₃: C,54.56; H,5.15; N,3.98. Found: C,44.97; H,3.22; N,3.94.

IR(KBr pellet, cm⁻¹): 3231, 2798, 1627, 1520, 1454

[α]²⁸_{D}=−25.9 (c=0.054, MeOH)<

### EXAMPLE 3

### Synthesis of (R)-6,7-dihydroxy-1-(α-naphthylmethyl)-1,2,3,4-tetrahydroisoquinoline hydrobromide salt

### (Step 1): Synthesis of N-(3,4-dimethoxyphenylethyl)(α-naphthyl)acetamide

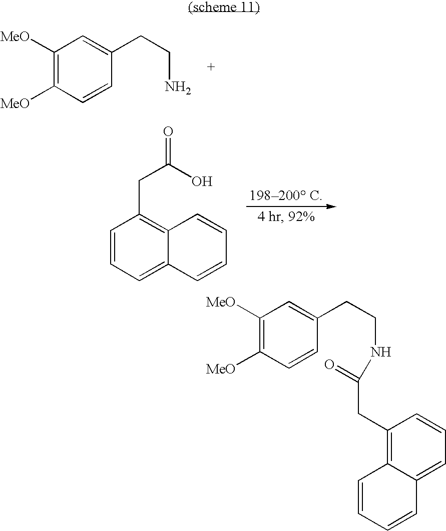

To 1-naphthylacetic acid (30.4 g, 0.163 mol) in a 500 mL round-bottom flask, 3,4-dimethoxyphenethylamine (27.5 mL, 0.163 mol) was added dropwise. The flask, equipped with a septum, was charged with nitrogen gas. Using the thermostat, the reaction solution was adjusted to the temperature of 198-200° C. and heated for 4 hours. Completion of the reaction was detected by thin film chromatography, and the reaction mixture was cooled to room temperature. Thereafter, the produced precipitate was dissolved in chloroform (100 mL). The chloroform solution was successively washed with 1 N HCl, 1 N NaHCO₃ and saturated brine, dried over anhydrous magnesium sulfate and filtered via the glass filter. The filtrate was concentrated under reduced pressure to give a yellowish solid. The solid was dissolved in a minimal amount of chloroform. Ether (400 mL) was added to the solution, with stirring, yielding a white solid. Such a solid was filtered via a Buchner funnel, followed by removing the solvent under reduced pressure, to give the desired compound, N-(3,4-dimethoxyphenethyl)(p-naphthyl)acetamide (52.3 g, 92%) as a white solid.

m.p=130° C.

R_{f}: 0.2 (hexane:ethyl acetate=2:1)

¹H-NMR(300 MHz, CDCl₃) δ 7.9-7.3(m,7H), 6.52(s,1H), 6.45(d,1H), 6.2(dd,1H), 5.25(s,2H), 4.0(s,2H), 3.85(s,2H) 3.75(s,3H), 3.38(q,H), 2.54(t,2H)

HRMs: m/z calcd for C₂₂H₂₃NO₂ (M+):349.17. Found: 349.13

Anal. calcd for C₂₂H₂₃NO₂: C,75.62; H,6.63; N,4.01. Found: C,75.64; H,6.61; N,4.03.

IR(KBr pellet, cm⁻¹): 3068, 2996, 2940, 1657, 1550, 1530,

### (Step 2): Synthesis of 6,7-dimethoxy-1-α-naphthylmethyl)-3,4-dihydroisoquinoline hydrochloride salt

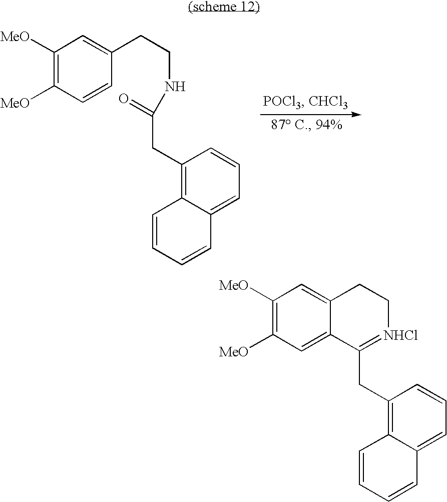

In a round-bottom flask, N-(3,4-dimethoxyphenethyl) (α-naphthyl) acetamide (50 g, 0.143 mol) was dissolved in chloroform (400 mL), to which POCl₃ (53.4 mL, 0.572 mol) was added dropwise. The flask, equipped with a reflux condenser, was charged with nitrogen gas. Using the thermostat, the reaction solution was adjusted to the temperature of 87° C. and refluxed for 33 hours. Completion of the reaction was detected by thin film chromatography, and the chloroform solvent was removed under reduced pressure. The produced light-green solid was dissolved in a minimal amount of chloroform, and added with distilled ethyl acetate (400 mL), with stirring, to precipitate the solid. The precipitated solid was filtered via a Buchner funnel and the solvent was removed under reduced pressure, to give the desired compound, iminium salt (49.4 g, 94%) as a light-green solid.

m.p=150° C.

R_{f}: 0.2 (hexane:ethyl acetate=0.5:1)

¹H-NMR(300 MHz, DMSO-d₆) δ 8.2(d,1H), 8.01(d,1H), 7.87(d,1H), 7.6(m,2H), 7.45(d,2H), 7.2(s,2H), 5.1(s,2H), 3.85(s,6H), 3.1(t,2H)

HRMs: m/z calcd for C₂₂H₂₂Cl NO₂ (M+): 367.13. Found: 368.49

Anal. calcd for C₂₂H₂₂ClNO₂: C,71.83; H,6.03; N,3.81. Found: C,66.71; H,6.39; N,2.98.

IR(KBr pellet, cm⁻¹): 3241, 2896, 1643, 1561, 1536, 1475

### (Step 3): Synthesis of (R)-6,7-dimethoxy-1-α-naphthylmethyl)-1,2,3,4-tetrahydroisoquinoline

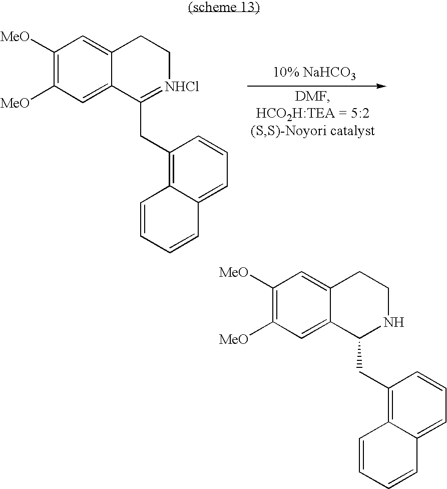

The iminium salt (15.0 g, 40.8 mmol) was dissolved in chloroform (70 mL), to which 10% aqueous NaHCO₃ solution (130 mL) was slowly added dropwise at 0° C., with stirring at 0° C. for 1 hour. The reaction mixture was extracted with chloroform (3×60 mL), and the combined organic layer was dried over anhydrous magnesium sulfate and filtered with the glass filter. The filtrate was concentrated under reduced pressure, to give 6,7-dimethoxy-1-(α-naphthylmethyl)-3,4-dihydroisoquinoline (13.2 g, 98%) as a pale yellow solid. The obtained imine was dissolved in DMF (80 mL), to which (S,S)—Ru (II) catalyst (0.233 g) was added and then distilled HCO₂H:TEA=5:2 (25 ml) were added thereto dropwise. The flask, charged with nitrogen gas, was equipped with a septum. Then, the reaction mixture was stirred for 12 hours. While completion of the reaction was detected by TLC, the reaction was terminated with 20% aqueous Na₂CO₃ solution. As such, 20% aqueous Na₂CO₃ solution was added to the extent of obtaining a slightly basic reaction mixture. Such a mixture was extracted with methylene chloride, and the organic layer was washed with saturated brine, dried over anhydrous magnesium sulfate and filtered via the glass filter. The filtrate was concentrated under reduced pressure, to give a crude product as a deep green liquid.

The amine was dissolved in acetic acid (40 mL) and 48% HBr (12 mL) was added thereto dropwise. The mixture was stirred for 2 hours to give a deep green precipitate to give an emulsion. Ether (100 ml) was added to the said precipitate, the emulsion was stirred for 1 hour, and the supernatant was discarded. Ether (200 ml) was added to the said emulsion. The said emulsion was stirred for 1 hour. The solid product was filtered and washed with ethyl acetate. The solvent was removed under reduced pressure to give ammonium salt as a light green solid. The said ammonium salt was dissolved in the solution prepared by mixing dichloromethane and methanol in a ratio of 5:1. n-hexane was added to the resultant solution to recrystallize a crystal (8.9 g). The crystal was dissolved in chloroform (90 ml), thereto 2N NaOH (70 ml) was added at 0° C. The aqueous layer was extracted with chloroform three times. The organic layer was washed with saturate brine, dried over MgSO₄, filtered and concentrated under reduced pressure to give amine (7.2 g). The said purification process was repeated one more times, to give a white solid free amine (6.8 g, 50%). Purity (98% or more) and ee value (99% or more) of the product was measured by use of HPLC (Daicel Chiralcel OD 4.6 mm×25 cm column: developing liquid-hexane:2-propanol:diethylamine=40:10:0.05; flowing rate—0.5 mL/min; retention time—30.7 min).

m.p=199° C.

R_{f}: 0.4 (ethanol:hexane=3:1)

¹H-NMR(500 MHz, CDCl₃) δ 8.25(d,1H), 7.85(d,1H), 7.78(d,1H), 7.45(dd,2H), 7.34(t,1H), 7.16(d,1H), 6.56(s,1H), 5.3(s,1H), 4.89(dd,1H), 4.32(dd,1H), 3.78(s,3H), 3.69(m,1H), 3.57(m,1H), 3.44(t,1H), 3.27(m,1H), 3.13(tt,1H)

HRMs: m/z calcd for C₂₂H₂₃NO₂ (M+):333.17. Found: 333.62

Anal. calcd for C₂₂H₂₃NO₂: C,79.25; H,6.95; N,4.20. Found: C,72.00; H,6.74; N,3.88.

IR(KBr pellet, cm⁻¹): 3434, 2940, 2793, 2553, 2467, 1617, 1530, 1479

[α]²⁹_{D}=−50.6 (c=0.063, CDCl₃)

### (Step 4): Synthesis of (R)-6,7-dimethoxy-1-α-naphthylmethyl)-1,2,3,4-tetrahydroisoguinoline hydrobromide salt

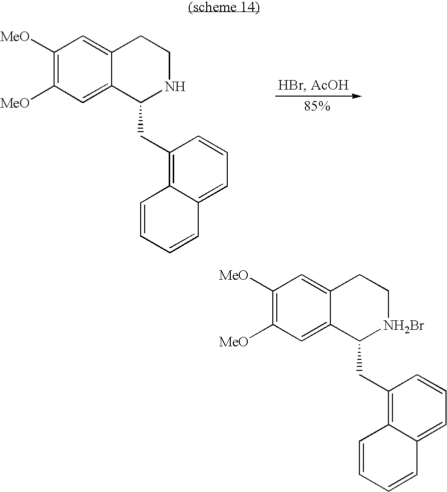

In a 1 L round-bottom flask, the amine (4.50 g, 13.5 mmol) was dissolved in acetic acid (20 mL), to which 48% HBr (4.5 mL) was added dropwise. Within 10 minute, an ammonium salt was produced, which was then added with ether (100 mL), stirred for 1 hour, washed with ethyl acetate two times, and filtered via a Buchner funnel. The solvent was removed under reduced pressure to give the desired compound, ammonium salt as a white solid. The said ammonium salt was dissolved in the solution prepared by mixing methanol and methylene chloride in a ratio of 1:5. hexane was added to the resultant solution. Thereafter, the resultant solution was grinded to give pure ammonium salt (4.77 g, 85%).

m.p=238° C.

¹H-NMR(300 MHz, DMSO-d₆) δ 8.25(d,1H), 8.05(d,1H), 8.01(d,1H), 7.64(m,2H), 7.61(t,1H), 7.45(d,1H), 6.88(s,1H), 6.03(s,1H), 4.87(s,1H), 3.84(s,6H), 3.8-3.0(m)

HRMs: m/z calcd for C₂₂H₂₄BrNO₂ (M+):413.10. Found: 412.42

Anal. calcd for C₂₂H₂₄BrNO₂: C,63.77; H,5.84; N,3.38. Found: C,62.03; H,6.17; N,3.45.

IR(KBr pellet, cm⁻¹): 3438, 2944, 2786, 2536, 1614, 1522, 1471

[α]²⁹_{D}=−69.0 (c=0.050, DMSO)

### (Step 5): Synthesis of (R)-6,7-dihydroxy-1-α-naphthylmethyl)-1,2,3,4-tetrahydroisoguinoline hydrobromide salt

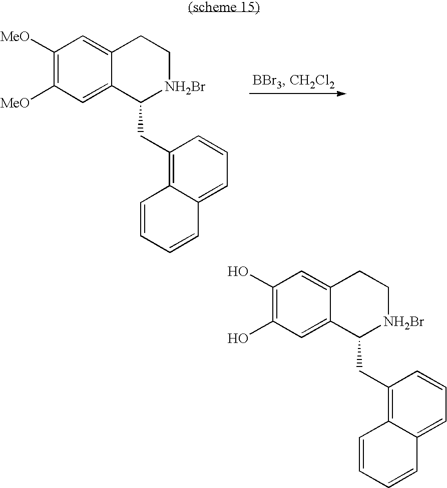

In a 500 mL round-bottom flask, the ammonium salt (4.77 g, 11.5 mmol) was dissolved in methylene chloride (80 mL). The flask was charged with nitrogen gas, to which BBr₃ (21.5 mL) was slowly added dropwise at −78° C. The reaction temperature was gradually increased to 0° C., and the reaction mixture was stirred for 3 hours. While the reaction completion was detected by NMR, the reaction was terminated with H₂O and ethanol. Then, the reaction mixture was stirred for 1 hour, washed with ethyl acetate two times, followed by filtering the produced precipitate through a Buchner funnel. The solvent was removed under reduced pressure, to give (R)-6,7-dihydroxy-1-α-naphthylmethyl)tetrahydroisoquinoline hydrobromide salt (3.47 g, 78%) as a white solid. Purity (98% or more) and ee value (99% or more) of the product were measured by use of HPLC (CHIREX 3020G-EO, Phenomenex Co., U.S.A., 4.6 mm×25 cm column: developing solvent-hexane:methylene chloride:trifluoroacetic acid/ethanol (1/20)=53:35:12; flowing rate—0.9 mL/min; retention time—9.9 min).

m.p=255° C.

R_{f}: 0.50 (3 drops of 28% aqueous ammonium hydroxide solution were added to a mixed developing-solvent of benzene:acetone:MeOH=5:4:2)

¹H-NMR(300 MHz, DMSO-d₆) δ 9.0-7.3(m,7H), 6.6(s,1H), 6.4(s,1H), 4.7(s,1H), 3.21(d,2H), 2.95(t,2H), 2.85(t,2H)

HRMs: m/z calcd for C₂₀H₂₀BrNO₂ (M+):385.07. Found: 485.46

Anal. calcd for C₂₀H₂₀BrNO₂: C,62.19; H,5.22; N,3.63. Found: C,62.21; H,5.26; N,3.66.

IR(KBr pellet, cm¹): 3419, 2935, 2788, 2559, 1632, 1535, 1415

[α]²⁹_{D}=−68.7 (c=0.048, MeOH)<

### EXAMPLE 4

### Synthesis (S)-6,7-dihydroxy-1-α-naphthylmethyl)-1,2,3,4-tetrahydroisoquinoline hydrobromide salt

Step 1 and step 2 were accomplished in the same procedure as ones of the said example 3.

### (Step 3): Synthesis of (S)-6,7-dimethoxy-1-α-naphthylmethyl)-1,2,3,4-tetrahydroisoquinoline

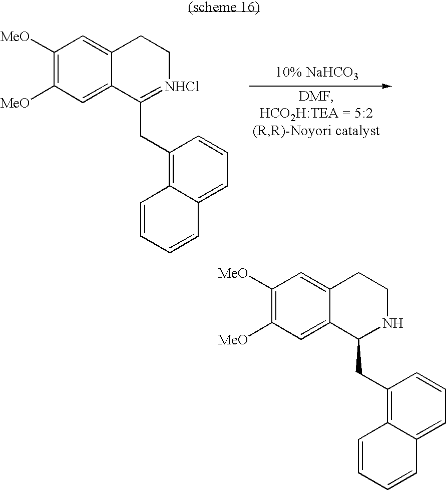

The iminium salt (15.0 g, 40.8 mmol) was dissolved in chloroform (70 mL), to which 10% aqueous NaHCO₃ solution (130 mL) was slowly added dropwise at 0° C., with stirring at 0° C. for 1 hour. The reaction mixture was extracted with chloroform (3×60 mL), and the combined organic layer was dried over anhydrous magnesium sulfate and filtered via the glass filter. The filtrate was concentrated under reduced pressure, to give 6,7-dimethoxy-1-(α-naphthylmethyl)-3,4-dihydroisoquinoline (13.5 g, 99%) as a pale yellow solid. The obtained imine was dissolved in DMF (80 mL), to which (R,R)—Ru (II) catalyst (0.234 g) was added and then distilled HCO₂H:TEA=5:2 (25 ml) was added thereto dropwise. The flask, charged with nitrogen gas, was equipped with a septum. Then, the reaction mixture was stirred for 12 hours. While completion of the reaction was detected by TLC, the reaction was terminated with 20% aqueous Na₂CO₃ solution. As such, 20% aqueous Na₂CO₃ solution was added to the extent of obtaining a slightly basic reaction mixture. Such a mixture was extracted with methylene chloride, and the organic layer was washed with a saturated brine, dried over anhydrous magnesium sulfate and filtered via the glass filter. The filtrate was concentrated under reduced pressure, to give a crude product as a deep green liquid.

The said amine was dissolved in acetic acid (45 mL) and 48% HBr (14 mL) was added thereto dropwise. The mixture was stirred for 2 hours to give a deep green precipitate. Ether (100 ml) was added to the said precipitate to give an emulsion. The emulsion was stirred for 1 hour, and the supernatant was discarded. Ether (200 ml) was added the said emulsion. The said emulsion was stirred for 1 hour. The solid product was filtered and washed with ethyl acetate. The solvent was removed under reduced pressure to give ammonium salt as a light green solid. The said ammonium salt was dissolved in the solution prepared by mixing dichloromethane and methanol in a ratio of 5:1. n-hexane was added to the resultant solution to recrystallize a crystal (8.4 g). The crystal was dissolved in chloroform (90 ml), thereto 2N NaOH (70 ml) was added at 0° C. The aqueous layer was extracted with chloroform three times. The organic layer was washed with saturate brine, dried over MgSO₄, filtered and concentrated under reduced pressure to give amine (7.0 g). The said purification process was repeated one more times, to give a white solid free amine (6.21 g, 46%). Purity (98% or more) and ee value (99% or more) of the product was measured by use of HPLC (Daicel Chiralcel OD 4.6 mm×25 cm column: developing liquid-hexane:2-propanol:diethylamine=40:10:0.05; flowing rate—0.5 mL/min; retention time—25.2 min).

m.p: 199° C.

R_{f}: 0.4 (ethanol:hexane=3:1)

¹H-NMR(300 MHz, CDCl₃) δ 8.25(d,1H), 7.85(d,1H), 7.78(d,1H), 7.45(dd,2H), 7.34(t,1H), 7.16(d,1H), 6.56(s,1H), 5.3(s,1H), 4.89(dd,1H), 4.32(dd,1H)), 3.78(s,3H), 3.69(m,3H), 3.57(m,3H), 3.44(t, 1H), 3.27(t, 1H), 3.13(tt, 1H)

### (Step 4): Synthesis of (S)-6,7-dimethoxy-1-α-naphthylmethyl)-1,2,3,4-tetrahydroisoguinoline hydrobromide salt

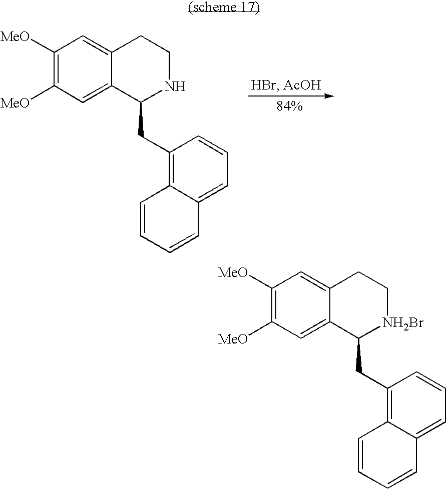

In a 300 ml round-bottom flask, the amine (6.21 g, 18.6 mmol) was dissolved in acetic acid (20 mL), to which 48% HBr (6.8 mL) was added dropwise. Within 10 minute, an ammonium salt was produced, to which ether (100 mL) was added. The solution was stirred for 1 hour, washed with ethyl acetate two times, and filtered via a Buchner funnel. The solvent was removed under reduced pressure to give the desired compound, ammonium salt as a white solid. The said ammonium salt was dissolved in the solution prepared by mixing methanol and methylene chloride in a ratio of 1:5. Hexane was added to the resultant solution. Thereafter, the resultant solution was grinded to give pure ammonium salt (6.46 g, 84%).

m.p=238° C.

¹H-NMR(300 MHz, DMSO-d₆) δ 8.25(d,1H), 8.05(d,1H), 8.01(d,1H), 7.64(m,2H), 7.61(t,1H), 7.45(d,1H), 6.88(s,1H), 6.03(s,1H), 4.87(s,1H), 3.84(s,6H), 3.8-3.0(m)

HRMs: m/z calcd for C₂₂H₂₄BrNO₂ (M+):413.10. Found: 412.42

Anal. calcd for C₂₂H₂₄BrNO₂: C,63.77; H,5.84; N,3.38. Found: C,62.03; H,6.17; N,3.45.

IR(KBr pellet, cm⁻¹): 3438, 2944, 2786, 2536, 1614, 1522, 1471

[α]²⁹_{D}=+69.0 (c=0.050, DMSO)

### (Step 5): Synthesis of (S)-6,7-dihydroxy-1-(α-naphthylmethyl)-1,2,3,4-tetrahydroisoguinoline hydrobromide salt

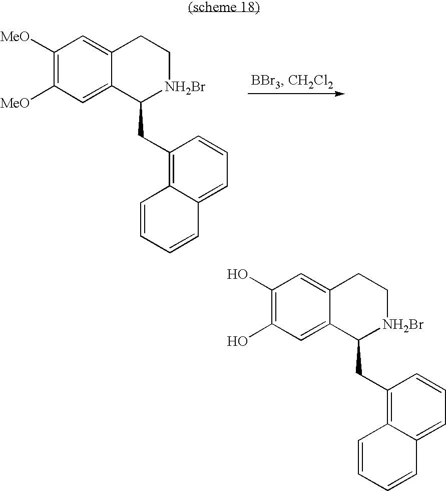

In a 500 mL round-bottom flask, the ammonium salt (6.46 g, 0.0156 mmol) was dissolved in methylene chloride (110 mL). The flask was charged with nitrogen gas, to which BBr₃ (30 mL) was slowly added dropwise at −78° C. The reaction temperature was gradually increased to 0° C. and the reaction mixture was stirred for 3 hours. While completion of the reaction was detected by NMR, the reaction was terminated with H₂O and ethanol. The reaction mixture was stirred for 1 hour, washed with ethyl acetate two times, and the produced precipitate was filtered via a Buchner funnel. The solvent was removed under reduced pressure, to give (S)-6,7-dihydroxy-1-(4-hydroxyphenylmethyl)tetrahydroisoquinoline hydrobromide salt as a white solid (4.71 g, 78%). Purity (98% or more) and ee value (99% or more) of the product were measured by use of HPLC (CHIREX 3020G-EO, Phenomenex Co., U.S.A., 4.6 mm×25 cm column: developing solvent-hexane:methylene chloride:trifluoroacetic acid/ethanol (1/20)=53:35:12; flowing rate—0.9 mL/min; retention time-11.9 min).

m.p=255° C.

R_{f}: 0.50 (3 drops of 28% aqueous ammonium hydroxide solution were added to a mixed developing-solvent of benzene:acetone:MeOH=5:4:2)

¹H-NMR(300 MHz, DMSO-d₆) δ 9.0-7.3(m,7H), 6.6(s, 1H), 6.4(s, 1H), 4.7(s,1H), 3.21(d,2H), 2.95(t,2H), 2.85(t,2H)

HRMs: m/z calcd for C₂₀H₂₀BrNO₂ (M+):385.07. Found: 485.46

Anal. calcd for C₂₀H₂₀BrNO₂: C,62.19; H,5.22; N,3.63. Found: C,62.21; H,5.26; N,3.66.

IR(KBr pellet, cm⁻¹): 3419, 2935, 2788, 2559, 1632, 1535, 1415

[α]²⁸_{D}=+66.76 (c=0.05, MeOH)

### (Step 6): Synthesis of (S)-6,7-dihydroxy-1-α-naphthylmethyl)-1,2,3,4-tetrahydroisoguinoline

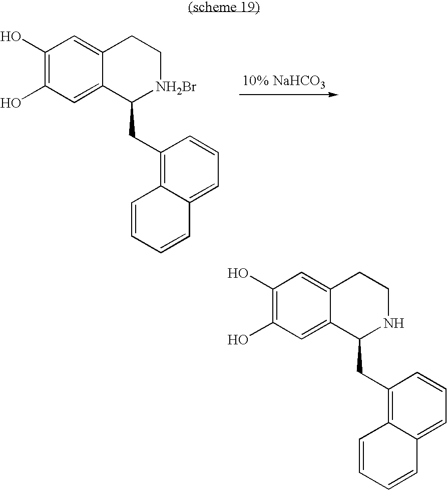

The ammonium salt (100 mg, 0.26 mmol) was dissolved in chloroform (3 mL) and methanol (3 ml), to which 10% aqueous NaHCO₃ solution (5 mL) was slowly added dropwise at 0° C., with stirring at 0° C. for 30 min. The reaction mixture was extracted with chloroform (3×10 mL), and the combined organic layer was washed with saturated brine, dried over anhydrous magnesium sulfate and filtered via the glass filter. The filtrate was concentrated under reduced pressure, to give 6,7-dihydroxy-1-α-naphthylmethyl)-1,2,3,4-tetrahydroisoquinoline (40 mg, 51%) as a light pink solid.

R_{f}: 0.46 (ethanol:hexane=3:1)

¹H-NMR(300 MHz, DMSO-d₆) δ 8.2-7.3(m, 7H), 6.63(s, 1H), 6.4(s, 1H), 4.6(br, 1H), 3.94(m, 1H), 3.58(d, 1H), 3.05(m, 2H), 2.41-2.71(m, 2H)

[α]²⁸_{D}=+20.4 (c=0.05, MeOH)<

### EXAMPLE 5

### Synthesis of (R)-6,7-dihydroxy-1-(β-naphthylmethyl)-1,2,3,4-tetrahydroisoquinoline hydrobromide salt

### (Step 1): Synthesis of N-(3,4-dimethoxyphenylethyl)(β-naphthyl)acetamide

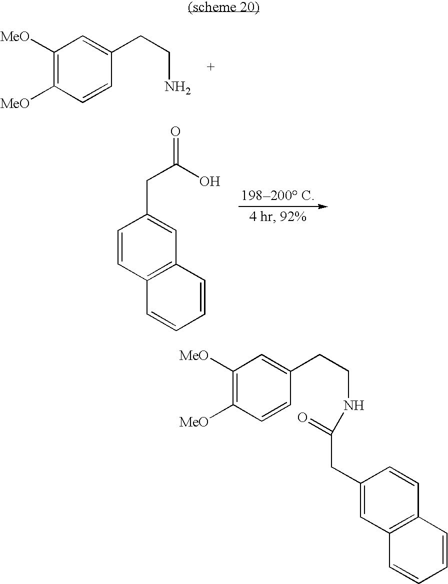

To a 250 mL round-bottom flask, 2-naphthylacetic acid (22.5 g, 0.121 mol) was added and then 3,4-dimethoxyphenethylamine (20.4 mL, 0.121 mol) was added thereto dropwise. The flask, equipped with a septum, was filled with nitrogen gas. Using the thermostat, the reaction solution was adjusted to the temperature of 198-200° C. and heated for 4 hours. Completion of the reaction was detected by thin film chromatography, and the reaction mixture was cooled to the room temperature. Then, the produced precipitate was dissolved in chloroform (250 mL). The chloroform solution was successively washed with 1 N HCl, 1 N NaHCO₃ and saturated brine, dried over anhydrous magnesium sulfate and filtered via the glass filter. The filtrate was concentrated under reduced pressure to give a yellowish solid, which was dissolved in a minimal amount of chloroform and ether (500 mL) was added thereto with stirring, yielding a white solid. Such a solid was filtered via a Buchner funnel and the solvent was removed under reduced pressure, to give the desired compound, N-(3,4-dimethoxyphenethyl)(β-naphthyl)acetamide (38.1 g, 92%) as a white solid.

m.p=135° C.

R_{f}: 0.2 (hexane:ethyl acetate=2:1)

¹H-NMR(300 MHz, CDCl₃) δ 7.9-7.3(m,7H), 6.7(br,1H), 6.55(s,3H), 3.85(s,3H), 3.8(s,3H) 3.7(s,2H), 3.75(q,2H), 2.62 (t,2H)

HRMs: m/z calcd for C₂₂H₂₃NO₂ (M+):349.17. Found: 349.19

Anal. calcd for C₂₂H₂₃NO₂: C,75.62; H,6.63; N,4.01. Found: C,75.63; H,6.64; N,4.07.

IR(KBr pellet, cm⁻¹): 3332, 2941, 1638, 1589, 1541, 1452

### (Step 2): Synthesis of 6,7-dimethoxy-1-(β-naphthylmethyl)-3,4-dihydroisoquinoline hydrochloride salt

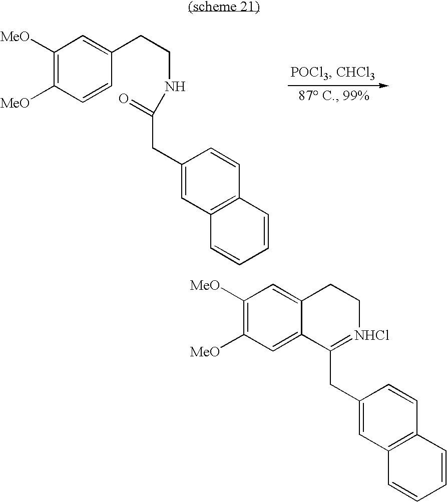

In a round-bottom flask, N-(3,4-dimethoxyphenylethyl)(β-naphthyl)acetamide (38 g, 0.109 mol) was dissolved in chloroform (300 mL), to which POCl₃ (40.5 mL, 0.435 mol) was added dropwise. The flask, equipped with a reflux condenser, was charged with nitrogen gas. Using the thermostat, the reaction solution was adjusted to the temperature of 87° C. and refluxed for 33 hours. Completion of the reaction was detected by thin film chromatography, and the chloroform solvent was removed under reduced pressure. The produced light-green solid was dissolved in a minimal amount of chloroform, and added with distilled ethyl acetate (400 mL), with stirring, to precipitate the solid. The precipitated solid was filtered through a Buchner funnel, followed by removing the solvent under reduced pressure, to give the desired compound, iminium salt (39 g, 99%) as a light-green solid.

R_{f}: 0.2 (hexane:ethyl acetate=0.5:1)

¹H-NMR(300 MHz, DMSO) δ 8.2-7.6(m, 6H), 7.05(s, 1H), 6.82(s, 1H), 6.61(s, 1H), 4.8(s, 2H), 3.85(s, 6H), 3.01(t, 2H), 2.65(t, 2H)

### (Step 3): Synthesis of (R)-6,7-dimethoxy-1-(β-naphthylmethyl)-1,2,3,4-tetrahydroisoguinoline

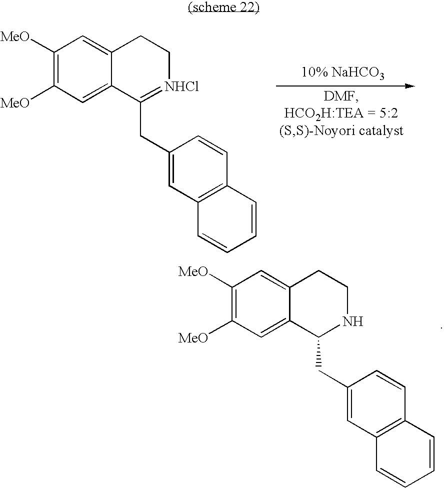

The iminium salt (15.0 g, 40.8 mmol) was dissolved in chloroform (70 mL), to which 10% aqueous NaHCO₃ solution (130 mL) was slowly added dropwise at 0° C., with stirring at 0° C. for 1 hour. The reaction mixture was extracted with chloroform (3×60 mL), and the combined organic layer was dried over anhydrous magnesium sulfate and filtered with the glass filter. The filtrate was concentrated under reduced pressure, to give 6,7-dimethoxy-1-(β-naphthylmethyl)-3,4-dihydroisoquinoline (13.1 g, 98%) as a pale yellow liquid. The obtained imine was dissolved in DMF (80 mL), to which (S,S)—Ru (II) catalyst (0.230 g) was added and then distilled HCO₂H:TEA=5:2 (25 ml) was added dropwise. The flask, charged with nitrogen gas, was equipped with a septum. Then, the reaction mixture was stirred for 12 hours. While completion of the reaction was detected by TLC, the reaction was terminated with 20% aqueous Na₂CO₃ solution. As such, 20% aqueous Na₂CO₃ solution was used to the extent of obtaining a slightly basic reaction mixture. Such a mixture was extracted with methylene chloride, and the organic layer was washed with saturated brine, dried over anhydrous magnesium sulfate and filtered via the glass filter. The filtrate was concentrated under reduced pressure, to give a crude product as a deep green liquid.

The said amine was dissolved in acetic acid (30 mL), to which 48% HBr (11 mL) was added dropwise. The mixture was stirred for 2 hours to give a deep green precipitate. Ether (100 ml) was added to the said precipitate to give an emulsion. The emulsion was stirred for 1 hour, and the supernatant was discarded. Ether (200 ml) was added to the said emulsion. The said emulsion was stirred for 1 hour. The solid product was filtered and washed with ethyl acetate. The solvent was removed under reduced pressure to give ammonium salt as a light green solid. The said ammonium salt was dissolved in the solution prepared by mixing dichloromethane and methanol in a ratio of 5:1. n-hexane was added to the resultant solution to recrystallize a crystal (6.1 g). The crystal was dissolved in chloroform (90 ml), therein 2N NaOH (70 ml) was added at 0° C. The aqueous layer was extracted with chloroform three times. The combined organic layer was washed with saturated brine, dried over MgSO₄, filtered and concentrated under reduced pressure to give amine (4.92 g). The said purification process was repeated one more times, to give a white solid free amine (4.80 g, 36%). purity (98% or more) and ee value (99% or more) of the product was measured by use of HPLC (Daicel Chiralcel OD 4.6 mm×25 cm column: developing liquid-hexane:2-propanol:diethylamine=40:10:0.05; flowing rate—0.5 mL/min; retention time—41.6 min).

m.p=208° C.

R_{f}: 0.4 (ethanol:hexane=3:1)

¹H-NMR(500 MHz, DMSO-d₆) δ 7.8-7.68(m, 3H), 7.63(s, 1H), 7.43(m, 2H), 7.36(dd, 1H), 6.55(s, 1H), 5.82(s, 1H), 4.82(dd, 1H), 3.87(dd, 1H), 3.79(s, 3H), 3.27(s, 3H), 3.39(t, 2H), 3.17(m, 1H), 3.11(s, 1H), 2.96(tt, 1H) 3.01(m, 2H)

Anal. calcd for C₂₂H₂₃BrNO₂: C,79.25; H,6.95; N,4.20. Found: C,71.75; H,6.82; N,3.92.

[α]²⁹_{D}=−33.1 (c=0.049, CDCl₃)

### (Step 4): Synthesis of (R)-6,7-dimethoxy-1-(β-naphthylmethyl)-1,2,3,4-tetrahydroisoguinoline hydrobromide salt

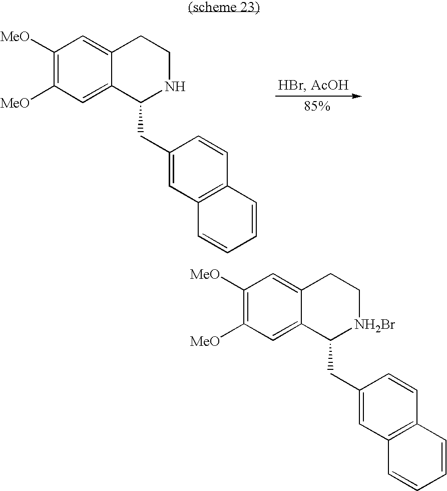

In a 300 ml round-bottom flask, the amine (4.80 g, 14.4 mmol) was added and dissolved in acetic acid (17 mL), to which 48% HBr (5.7 mL) was added dropwise. Within 10 minute, an ammonium salt was produced, to which ether (100 mL) was added, stirred for 1 hour, washed with ethyl acetate two times and filtered via a Buchner funnel. The solvent was removed under reduced pressure, to give the desired compound, ammonium salt as a white solid. The said ammonium salt was dissolved in the solution prepared by mixing methanol and methylene chloride in a ratio of 1:5. Hexane was added to the resultant solution. The resultant solution was grinded to give pure ammonium salt (5.1 g, 85%).

m.p=235° C.

¹H-NMR(300 MHz, DMSO-d₆) δ 8.0-7.75(m, 4H), 7.4-7.6(m, 3H), 6.8(s, 1H), 6.6(s, 1H), 4.8(s, 1H), 3.85(s, 6H) 3.0-3.6(m)

Anal. calcd for C₂₂H₂₄BrNO₂: C,63.77; H,5.84; N,3.38. Found: C,63.71; H,5.91; N,3.41.

HRMs: m/z calcd for C₂₂H₂₄BrNO₂ (M+):413.10. Found: 411.90

IR(KBr pellet, cm⁻¹): 3421, 2733, 2602, 2465, 1611, 1525, 1439

### (Step 5): Synthesis of (R)-6,7-dihydroxy-1-(β-naphthylmethyl)-1,2,3,4-tetrahydroisoguinoline hydrobromide salt

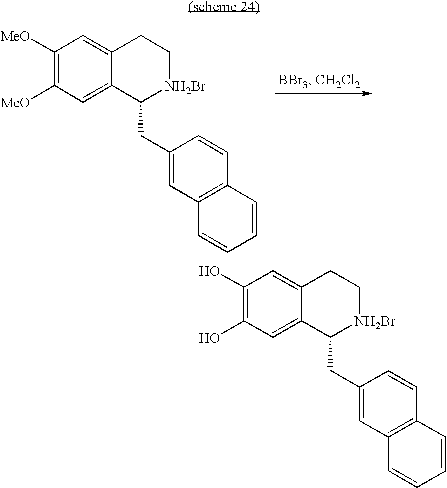

In a 500 mL round-bottom flask, the ammonium salt (5.1 g, 12.3 mmol) was dissolved in methylene chloride (70 mL). The flask was charged with nitrogen gas, to which BBr₃ (23.7 mL) was slowly added dropwise at −78° C. The reaction temperature was gradually increased to 0° C., and the reaction mixture was stirred for 3 hours. While completion of the reaction was detected by NMR, the reaction was terminated with H₂O and ethanol. Then, the reaction mixture was stirred for 1 hour, washed with ethyl acetate two times, followed by filtering the produced precipitate through a Buchner funnel. The solvent was removed under reduced pressure, to give (R)-6,7-dihydroxy-1-(β-naphthylmethyl)tetrahydroisoquinoline hydrobromide salt (4.05 g, 89%) as a white solid. Purity (98% or more) and ee value (99% or more) of the product were measured by use of HPLC (CHIREX 3020G-EO, Phenomenex Co., U.S.A., 4.6 mm×25 cm column: developing solvent-hexane:methylene chloride:trifluoroacetic acid/ethanol (1/20)=53:35:12; flowing rate—0.9 mL/min; retention time—9.9 min).

m.p=244° C.

R_{f}: 0.50 (3 drops of 28% ammonium hydroxide aqueous solution were added to a mixed developing-solvent of benzene:acetone:MeOH=5:4:2)

¹H-NMR(300 MHz, DMSO-d₆) δ 9.35(br, 1H), 9.06(br, 1H), 8.84(br, 1H), 7.1(d, 2H), 6.7(d, 2H), 6.5(d, 2H), 2.79-3.17(m, 6H)

HRMs: m/z calcd for C₂₀H₂₀BrNO₂ (M+):385.07. Found: 385.08

Anal. calcd for C₂₀H₂₀BrNO₂: C,62.19; H,5.22; N,3.63. Found: C,62.19; H,5.22; N,3.67.

IR(KBr pellet, cm⁻¹): 3162, 2966, 2800, 1628, 1541, 1441

[α]²⁹_{D}=+10.4 (c=0.051, MeOH)<

### EXAMPLE 6

### Synthesis of (S)-6,7-dihydroxy-1-(β-naphthylmethyl)-1,2,3,4-tetrahydroisoquinoline hydrobromide salt

step 1 and step 2 were accomplished in the same procedure as ones of the said example 5

### (Step 3): Synthesis of (S)-6,7-dimethoxy-1-(β-naphthylmethyl)-1,2,3,4-tetrahydroisoquinoline

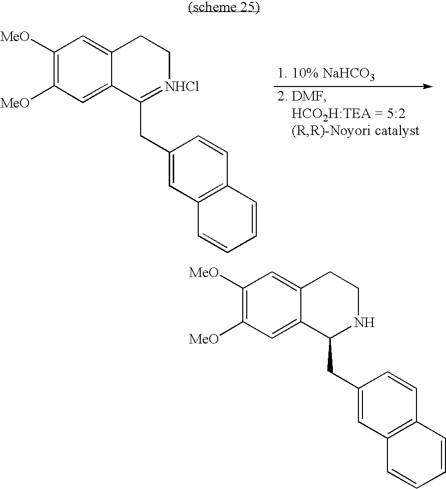

The iminium salt (30.0 g, 0.082 mol) was dissolved in chloroform (150 mL), to which 10% aqueous NaHCO₃ solution (130 mL) was slowly added dropwise at 0° C., with stirring at 0° C. for 1 hour. The reaction mixture was extracted with chloroform (3×60 mL), and the combined organic layer was dried over anhydrous magnesium sulfate and filtered with the glass filter. The filtrate was concentrated under reduced pressure, to give 6,7-dimethoxy-1-(β-naphthylmethyl)-3,4-dihydroisoquinoline (25 g, 98%) as a pale yellow liquid. The obtained imine was dissolved in DMF (250 mL), to which (R,R)—Ru (II) catalyst (0.470 g) was added and then distilled HCO₂H:TEA=5:2 (50 ml) was added thereto dropwise. The flask, charged with nitrogen gas, was equipped with a septum. Then, the reaction mixture was stirred for 12 hours. While completion of the reaction was detected by TLC, the reaction was terminated with 20% aqueous Na₂CO₃ solution. As such, 20% aqueous Na₂CO₃ solution was used to the extent of obtaining a slightly basic reaction mixture. Such a mixture was extracted with methylene chloride, and the organic layer was washed with saturated brine, dried over anhydrous magnesium sulfate and filtered via the glass filter. The filtrate was concentrated under redeuced pressure, to give a crude product as a deep green liquid.

The said amine was dissolved in acetic acid (50 mL), to which 48% HBr (16 mL) was added dropwise. The mixture was stirred for 2 hours to give a deep green precipitate. Ether (200 ml) was added to the said precipitate to give an emulsion, the emulsion was stirred for 1 hour, and the supernatant was discarded. Ethyl acetate (200 ml) was added the said emulsion. The said emulsion was stirred for 1 hour. The solid product was filtered and washed with ethyl acetate. The solvent was removed under reduced pressure to give ammonium salt as a light green solid. The said ammonium salt was dissolved in the solution prepared by mixing dichloromethane and methanol in a ratio of 5:1. n-hexane was added to the resultant solution to recrystallize a crystal (16.5 g). The crystal was dissolved in chloroform (180 ml), thereto 2N NaOH (140 ml) was added at 0° C. The aqueous layer was extracted with chloroform three times. The combined organic layer was washed with saturated brine, dried over MgSO₄, filtered and concentrated under reduced pressure to give amine (13.2 g). The said purification process was repeated one more times, to give a white solid free amine (12.6 g, 50%). The purified amine was measured for purity (98% or more) and ee value (99% or more) by use of HPLC (Daicel Chiralcel OD 4.6 mm×25 cm column: developing liquid-hexane:2-propanol:diethylamine=40:10:0.05; flowing rate-0.5 mL/min; retention time—34.1 min).

m.p=208° C.

R_{f}: 0.4 (ethanol:hexane=3:1)

¹H-NMR(500 MHz, DMSO-d₆) δ 7.8-7.68(m, 3H), 7.63(s, 1H), 7.43(m, 2H), 7.36(dd, 1H), 6.55(s, 1H), 5.82(s, 1H), 4.82(dd, 1H), 3.87(dd, 1H), 3.79(s, 3H), 3.27(s, 3H), 3.39(t, 2H), 3.17(m, 1H), 3.11(s, 1H), 2.96(tt, 1H) 3.01(m, 2H)

Anal. calcd for C₂₂H₂₃BrNO₂: C,79.25; H,6.95; N,4.20. Found: C,71.75; H,6.82; N,3.92.

### (Step 4): Synthesis of (S)-6,7-dimethoxy-1-(β-naphthylmethyl)-1,2,3,4-tetrahydroisoquinoline hydrobromide salt

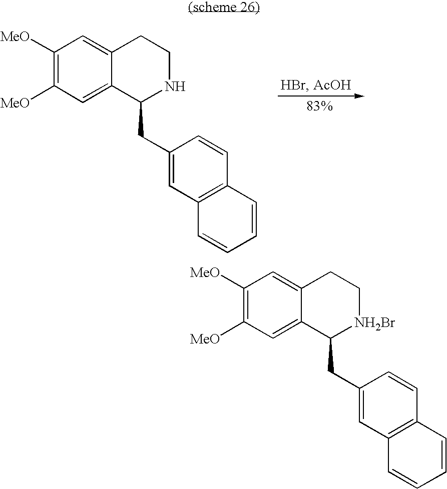

In a 1 L round-bottom flask, the amine (12.6 g, 37.8 mmol) was added and dissolved in acetic acid (38 mL), to which 48% HBr (12.5 mL) was added dropwise. Within 10 minute, an ammonium salt was produced, which was then added with ether (300 mL), stirred for 1 hour, washed with ethyl acetate two times and filtered via a Buchner funnel. The solvent was removed under reduced pressure, to give the desired compound, ammonium salt as a white solid. The said ammonium salt was dissolved in the solution prepared by mixing methanol and methylene chloride in a ratio of 1:5. Hexane was added to the resultant solution. The resultant solution was grinded to give pure ammonium salt (13.3 g, 85%).

m.p=235° C.

¹H-NMR(300 MHz, DMSO-d₆) δ 8.0-7.75(m, 4H), 7.4-7.6(m, 3H), 6.8(s, 1H), 6.6(s, 1H), 4.8(s, 1H), 3.85(s, 6H), 3.0-3.6(m)

Anal. calcd for C₂₂H₂₄BrNO₂: C,63.77; H,5.84; N,3.38. Found: C,63.71; H,5.91; N,3.41.

HRMs: m/z calcd for C₂₂H₂₄BrNO₂ (M+):413.10. Found: 411.90

IR(KBr pellet, cm⁻¹): 3421, 2733, 2602, 2465, 1611, 1525, 1439

### (Step 5): Synthesis of (S)-6,7-dihydroxy-1-(β-naphthylmethyl)-1,2,3,4-tetrahydroisoguinoline hydrobromide salt

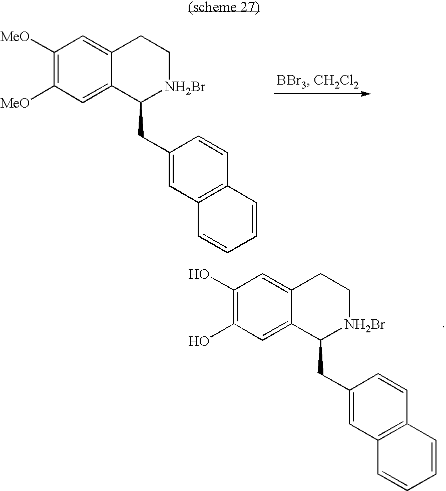

In a 500 mL round-bottom flask, the ammonium salt (6.5 g, 15.7 mmol) was dissolved in methylene chloride (100 mL). The flask was charged with nitrogen gas, to which BBr₃ (31.1 mL) was slowly added dropwise at −78° C. The reaction temperature was gradually increased to 0° C., and the reaction mixture was stirred for 3 hours. While the reaction completion was detected by NMR, the reaction was terminated with H₂O and ethanol. Then, the reaction mixture was stirred for 1 hour, washed with ethyl acetate two times, followed by filtering the produced precipitate through a Buchner funnel. The solvent was removed under reduced pressure, to give (S)-6,7-dihydroxy-1-(β-naphthylmethyl)tetrahydroisoquinoline hydrobromide salt (5.40 g, 89%) as a white solid. Purity (98% or more) and ee value (99% or more) of the product were measured by use of HPLC (CHIREX 3020G-EO, Phenomenex Co., U.S.A., 4.6 mm×25 cm column: developing solvent-hexane:methylene chloride:trifluoroacetic acid/ethanol (1/20)=53:35:12; flowing rate—0.9 mL/min; retention time—11.4 min).

m.p 244° C.

R_{f}: 0.50 (3 drops of 28% aqueous ammonium hydroxide solution were added to a mixed developing-solvent of benzene:acetone:MeOH=5:4:2)

¹H-NMR(300 MHz, DMSO-d₆) δ 9.35(br, 1H), 9.06(br, 1H), 8.84(br, 1H), 7.1(d, 2H), 6.7(d, 2H), 6.5(d, 2H), 2.79-3.17(m, 6H)

HRMs: m/z calcd for C₂₀H₂₀BrNO₂ (M+):385.07. Found: 385.08

Anal. calcd for C₂₀H₂₀BrNO₂: C,62.19; H,5.22; N,3.63. Found: C,62.19; H,5.22; N,3.67.

IR(KBr pellet, cm⁻¹): 3162, 2966, 1628, 1541, 1441

[α]²⁹_{D}=−10.7 (c=0.053, MeOH)<

### EXPERIMENTAL EXAMPLE

The experiment confirming the pharmacological effect of the compounds of the present invention was accomplished as shown in the below. Also, all the compounds of the present invention were used as hydrobromide salt.

### Experimental Example 1

Assay for Contractile Force and Heart Rates of Auricle Muscles Excised from Rats

Immediately after Sprague-Dawley rats (female or male) were anaesthetized with pentobarbital sodium (50 mg/kg, i.m.), the thorax was opened to excise the heart. The heart was submerged in Krebs solution, and left auricle muscle and right auricle muscle were then isolated. The left auricle muscle was placed in an organ bath for recording of contractile force, and applied with electrical field stimulation (voltage 10% higher than threshold voltage, 1 Hz frequency, 5 ms duration). In the case of right auricle muscle, which contracts spontaneously, the number of continuous beats was recorded. In this experiment, to measure contractile fore of the two auricle muscles, the organ bath was warmed to 37° C., and as a physiological solution, Krebs solution (119.8 mM NaCl, 4.6 mM KCl, 2.5 mM CaCl₂, 1.2 mM MgCl₂, 25 mM NaHCO₃, 1.2 mM KH₂PO₄, 10 mM glucose, 1 mM EDTA, pH 7.4) was used. The Krebs solution (pH 7.4) was continuously saturated with a gas mixture of 95% O₂ and 5% CO₂. The auricle muscle was under a tension of 1 g, and Krebs solution was exchanged with fresh solution at intervals of 20 minutes while equilibrium was maintained during a recording period of 60 minutes.

Conclusion:

As shown in the Tables 1a and lb, below, R-enantiomer represented by the formula 4 increased the contractile force and heart rates of auricle muscles, however effect of the said R-enantiomer was less than one tenth of S-enantiomer represented by the formula 3. R-enantiomers represented by the formula 6 or 8 have no effect to the contractile force and heart rates of auricle muscles, however, S-enantiomer represented by the formula 5 or 7 strongly increased contractile force of the auricle muscles and thus strongly stimulated their beating. Also, the racemic mixture increased the contractile force and heart rates of auricle muscles, weakly than S-enantiomer did, or strongly than R-enantiomer did.

TABLE 1a
Comparison of Contractile Force of Auricle Muscles exposed to Enantiomers (vehicle control:100%)

| | | | Racemic mixture | | | Racemic mixture | | | Racemic mixture |
|---|---|---|---|---|---|---|---|---|---|
| Concentration | Formula | Formula | of formula 3 | Formula | Formula | of formula 5 | Formula | Formula | of formula 7 |
| (M) | 3 | 4 | and formula 4 | 5 | 6 | and formula 6 | 7 | 8 | and formula 8 |
| 1 × 10⁻⁷ | 140 | 100 | 115 | 200 | 100 | 140 | 120 | 100 | 110 |
| 3 × 10⁻⁷ | 180 | 100 | 130 | 280 | 87 | 200 | 170 | 100 | 140 |
| 1 × 10⁻⁶ | 250 | 130 | 180 | 450 | 75 | 310 | 250 | 100 | 180 |
| 3 × 10⁻⁶ | 290 | 160 | 240 | 550 | 50 | 360 | 315 | 100 | 220 |
| 1 × 10⁻⁵ | 300 | 200 | 260 | 630 | 50 | 400 | 350 | 100 | 250 |
| 3 × 10⁻⁵ | 300 | 250 | 280 | 660 | 50 | 430 | 380 | 105 | 270 |
| 1 × 10⁻⁴ | 300 | 300 | 300 | 700 | 50 | 490 | 380 | 105 | 310 |

TABLE 1b
Comparison of Heart Rates of Auricle Muscles exposed to Enantiomers (vehicle control:100%)

| | | | Racemic mixture | | | Racemic mixture | | | Racemic mixture |
|---|---|---|---|---|---|---|---|---|---|
| Concentration | Formula | Formula | of formula 3 | Formula | Formula | of formula 5 | Formula | Formula | of formula 7 |
| (M) | 3 | 4 | and formula 4 | 5 | 6 | and formula 6 | 7 | 8 | and formula 8 |
| 1 × 10⁻⁷ | 130 | 100 | 110 | 150 | 100 | 115 | 150 | 100 | 110 |
| 3 × 10⁻⁷ | 140 | 100 | 110 | 162.5 | 93.75 | 115 | 160 | 100 | 120 |
| 1 × 10⁻⁶ | 145 | 110 | 115 | 175 | 93.75 | 120 | 165 | 100 | 125 |
| 3 × 10⁻⁶ | 150 | 115 | 125 | 178 | 94 | 120 | 170 | 100 | 135 |
| 1 × 10⁻⁵ | 180 | 120 | 140 | 180 | 85 | 130 | 175 | 105 | 140 |
| 3 × 10⁻⁵ | 200 | 130 | 160 | 187 | 75 | 130 | 180 | 105 | 140 |
| 1 × 10⁻⁴ | 250 | 140 | 200 | 190 | 71 | 135 | 180 | 105 | 140 |

### Experimental Example 2

Effect of Enantiomers on Rat Thoracic Aortas

Experimental group was divided into two groups, group containing endothelial cells of blood vessel and group not containing. Blood vessel of thoracic aortas was induced to contract by treatment with 0.1 μM of phenyleprine under tension of 1 g. Upon arriving at equilibrium, the enantiomers were added to an organ bath in a cumulative manner. Relaxation of thoracic aortas was recorded using a physiograph (Grass P7), calculating their response to the compounds with consideration of their added amount.

Conclusion:

As shown in the following Table 2, it was found that all of the compounds represented by the formulas 3, 4, 5, 6, 7 and 8 induced relaxation of contracted blood vessels in a concentration-dependant manner. In addition, all of the S-enantiomers (the compounds represented by formulas 3, 5 and 7) showed a stronger vasodilation effect than the R-enantiomers (the compounds represented by formulas 4, 6 and 8). However, the racemic mixture showed vasodilation effect, weaker than S-enetiomer did, or stronger than R-enantiomer did.

TABLE 2
Concentration of Compounds Showing Relaxation Response of 50%
(IC₅₀) given as Log Scale

| Compound | IC₅₀ (E+) | IC₅₀ (E−) |
|---|---|---|
| Formula 3 | 9.17 × 10⁻⁷ M | 4.3 × 10⁻⁶ M |
| Formula 4 | 4.64 × 10⁻⁶ M | 7.7 × 10⁻⁶ M |
| Racemic mixture of | 9.81 × 10⁻⁶ M | 5.61 × 10⁻⁶ M |
| formula 3 and formula 4 | | |
| Formula 5 | 7.14 × 10⁻⁷ M | 5.50 × 10⁻⁶ M |
| Formula 6 | 1.28 × 10⁻⁶ M | 8.81 × 10⁻⁶ M |
| Racemic mixture of | 1.05 × 10⁻⁷ M | 6.41 × 10⁻⁶ M |
| formula 5 and formula 6 | | |
| Formula 7 | 7.57 × 10⁻⁷ M | 3.14 × 10⁻⁶ M |
| Formula 8 | 1.95 × 10⁻⁶ M | 7.54 × 10⁻⁶ M |
| Racemic mixture of | 8.75 × 10⁻⁶ M | 5.15 × 10⁻⁵ M |
| formula 7 and formula 8 | | |

### Experimental Example 3

Effects of Enantiomers on Nitric Oxide Production in Macrophage Cell, Stimulated by LPS

RAW 264.7 macrophage cells were plated on 100 mm culture dishes at a density of 1-2×10⁵ cells. Thereafter, the said macrophage cell was incubated in DMEM medium containing 10% fetal calf serum (heat-inactivated), 100 U/ml penicillin, and 100 mg/ml streptomycin in a CO₂ incubator. The said macrophage cell was incubated therein confluently. The said incubated macrophage cells were transferred to serum-free DMEM medium, and further incubated therein for about 24 hours. Thereafter, macrophage cells were stimulated with LPS (1 μg/ml) for about 8 hours. (In this progress, experimental group was divided into two groups, group to which the compound was administered, and group to which the compound was not.) Then, nitrite quantification was achieved by performing calorimetric reaction for produced nitrite with Griess reagent and then measuring absorbance at 550 nm, while using NaNO₂ as a standard. The results are given in Table 3, below.

Conclusion:

As shown in the Table 3, NO produced in group treated with only LPS was 37.2 μM. However, when the said macrophage cell was treated with the various concentration (10, 50, and 100 μM) of compound represented by the formula 3, a S-enantiomer, the production of NO was reduced to 30.5, 22.3, and 18.2 μM, respectively, in inverse proportion to added concentration of the compound. Also, the compound represented by formula 4, R-enantiomer inhibited production of NO weakly than S-enantiomer did. Also, the racemic mixture of the compound represented by the formula 4 inhibited production of NO, weakly than S-enantiomer did or strongly than R-enantiomer did. And another S-enantiomer, the compound represented by formula 5 and 7 strongly inhibited production of NO, in inverse proportion to added concentration of the compounds. However, another R-enantiomer, the compound represented by formula 6 and 8 never or slightly inhibited production of NO. The racemic mixture of the respective compound inhibited production of NO, weakly than S-enantiomer did of strongly than R-enantiomer did.

TABLE 3
Effect of Enantiomers on NO production by LPS in macrophage cell

| | | | Formula | | | Formula | | | Formula |
|---|---|---|---|---|---|---|---|---|---|
| | | | 3/formula | | | 5/formula | | | 7/formula |
| | Formula | Formula | 4 (racemic | Formula | Formula | 6 (racemic | Formula | Formula | 8 (racemic |
| Concentration | 3 | 4 | mixture) | 5 | 6 | mixture) | 7 | 8 | mixture) |
| Control | 2.9 | 2.7 | 2.9 | 2.73 | 4.12 | 3.12 | 3.15 | 2.53 | 3.08 |
| LPS | 37.2 | 28.4 | 38.4 | 39 | 39 | 31 | 43.53 | 47.25 | 45 |
| 10 | 30.5 | 20.1 | 21.3 | 21.3 | 36 | 31 | 31.2 | 46 | 45 |
| 50 | 22.3 | 10.3 | 14.3 | 14.8 | 34 | 28 | 15.2 | 42 | 40 |
| 100 | 18.2 | 8.2 | 15.2 | 14.2 | 38 | 26 | 9.4 | 40 | 39 |

### Experimental Example 4

Effects of Enantiomers on Expression of iNOS Stimulated by LPS in Macrophage Cell by Western Blotting Analysis

In order to investigate whether inhibition of NO production by the enantiomer was derived from inhibition of expression of iNOS gene shown in the above experimental example 3, Western blotting analysis was performed. And the result was shown in FIG. 1.

Macrophage cells (RAW 264.7 cell) were plated on 100 mm culture dishes at a density of 1-2×10⁵ cells and incubated in DMEM medium containing 10% fetal calf serum (heat-inactivated), 100 U/ml penicillin, and 100 mg/ml streptomycin in a CO₂ incubator. When being grown to complete confluence, macrophage cell was inoculated in serum-free DMEM medium, and further incubated for about 24 hours. Thereafter, macrophage cells were stimulated in the presence of LPS (1 μg/ml) and each of the compounds represented by the formulas 3 to 8 for about 12 hours. Total protein was isolated from the macrophage cells. The isolated protein was quantified by using Bradford method. 10 μg of isolated protein was loaded in SDS-PAGE electroporesis gel. The said gel was transferred to PVDF membrane. Anti-iNOS antibody was added to the said gel, thereafter disposed at 4° C. overnight. Thereafter, secondary antibody was added to the said gel, reacted for 1 hour at the room temperature and sensitized using ECL. Expression of protein was quantified, compared to the expressed Actin.

As shown in FIG. 1, expression of iNOS protein was reduced by the compounds represented by the formulas 3 to 8. And S-enantiomer, the compound represented by formula 3, 5 and 7 inhibited expression of iNOS protein strongly than R-enantiomer, the compound represented by formula 4, 6 and 8 did. Also, S-enantiomer reduced expression of iNOS protein strongly than the racemic mixture corresponding to the said S-enantiomer did. Racemic mixture has inhibitory effect on expression of iNOS protein, where the effect is between one of R-enantiomer and S-enantiomer.

### Experimental Example 5

Inhibitory Effect of Enantiomers on Platelet Aggregation

Rats (Crj:CD(SD); 250±20 g) were used to test sample.

Male rat (weighing 200±20 g) was anaesthetized with ether. The blood (nine volumes) was collected from heart using a plastic syringe containing 2.2% sodium citrate (one volume). The collected blood was centrifuged at 200×g for 10 minutes to give supernatant platelet-rich plasma (PRP). The residue was centrifuged again at 700×g for 30 minutes, giving platelet-poor plasma (PPP) for experiment. We used a platelet analyzer to count the number of platelet of the PRP. PRP was diluted with physiological saline solution to prepare solution containing platelets in an amount of 400-450×10⁶/ml. The inhibitory effects of the compound represented by formulas 3 to 8 was evaluated by platelet aggregation analyzer, and the inhibitory effect of each compound is given as % below. That is, after PRP was incubated for 3 minutes at 37° C., the compounds or a carrier was added thereto, and after 1 min, platelet aggregation-inducing agent such as ADP, collagen, arachidonic acid (AA), U46619 or epinephrine was added thereto, followed by investigation of turbidity caused by aggregation of platelets.
% Inhibition=A−B/A×100 [arithmetic formula 1]
(A: Platelet aggregation rate in group treated with an aggregation-inducing agent;
B: Platelet aggregation rate in group treated with both an aggregation-inducing agent and the enantiomers simultaneously)

Conclusion;

The inhibitory effect of the enantiomers on platelet aggregation induced by ADP, collagen, epinephrine, AA or U46619 in rat PRP was analyzed. The results showing the inhibitory effect of the compounds represented by the formulas 3, 4, 5, 6, 7 to 8 on platelet aggregation are given in Table 4, below. The compounds represented by the formulas 3 and 4 did not inhibit platelet aggregation induced by ADP even at the concentration as high as 1×10⁻³ M. The compounds, represented by the formulas 5, 6, 7 and 8, showed very mild inhibitory effects on platelet aggregation induced by ADP with the IC₅₀ of 2.4˜5.4×10⁻⁴ M. There were no large difference in the inhibitory effects between R- and S-configurations of the compounds. On platelet aggregation induced by collagen, all of the enantiomers showed low inhibitory effects with IC₅₀ of 6.9˜45×10⁻⁵ M, which is slightly stronger than that on platelet aggregation induced by ADP, where there is no large difference in the inhibitory effect between R- and S-configurations. On platelet aggregation induced by AA, all of the enantiomers showed much stronger inhibitory effects with IC₅₀ of 5.6˜11×10⁻⁶ M, than that on platelet aggregation induced by ADP and collagen, where S-configurations represented by the formulas 3, 5 and 7 had inhibitory effects 1.2˜2 times as strong as R-configurations represented by the formulas 4, 6 and 8 did. On platelet aggregation induced by U46619, all of the enantiomers showed much stronger inhibitory effects than that on platelet aggregation induced by ADP and collagen, where there is no large difference in the inhibitory effect between R- and S-configurations. On platelet aggregation induced by epinephrine, all of the enantiomers showed much stronger inhibitory effects with IC₅₀ of 1.5×10⁻⁵ to 3.8×10⁻⁷ M, than that on platelet aggregation induced by ADP, collagen, AA or U46619, where S-configurations represented by the formulas 3, 5 and 7 had inhibitory effects stronger than R-configurations represented by the formulas 4, 6 and 8 did. That is, when rat PRP was treated with epinephrine, the compound of the formula 3 (IC₅₀: 1.6×10⁻⁶ M) had inhibitory effect of platelet aggregation about nine times as strong as the compound of the formula 4(IC₅₀: 1.4×10⁻⁵ M) did, the compound of the formula 5 (IC₅₀: 3.8×10⁻⁷ M) had inhibitory effect of platelet aggregation about six times as strong as the compound of formula 6 (IC₅₀: 2.4×10⁻⁶ M) did, and the compound of the formula 7 (IC₅₀: 9.0×10⁻⁷ M) had inhibitory effect of platelet aggregation about seventeen times as strong as the compound of the formula 8 (IC₅₀: 1.5×10⁻⁵ M) did, where there is a great difference in the inhibitory effect between the compounds represented by the formulas 7 and 8. In addition to, the racemic mixture of the compounds represented by formulas 3, 5 and 7, S-LO enantiomer, that is RS-configuration (formula 3+formula 4, formula 5+formula 6, formula 7+formula 8, respectively) showed IC₅₀ of 7.2×10⁻⁶, 1.7×10⁻⁶ and 6.3×10⁻⁶ M, respectively. (Yun-Choi, H. S. et al., Planta Med. 67, 619-622, 2001; and Thromb. Res., 104, 249-225, 2001). S-enantiomer had inhibitory effect of platelet aggregation about five times as strong as the racemic mixture did

| Platelet | | | | | |
|---|---|---|---|---|---|
| aggregation | | | | | |
| inducing | IC₅₀ (M) | | | | |
| agent | ADP^{a} | collagen^{b} | Epinephrine^{c,f} | AA^{d,f} | U46619^{e,f} |
| Formula 3 | >1.0 × 10⁻³ | 4.5 × 10⁻⁴ | 1.6 × 10⁻⁶ | 9.1 × 10⁻⁶ | 2.5 × 10⁻⁵ |
| Formula 4 | 1.0 × 10⁻³ | 3.0 × 10⁻⁴ | 1.4 × 10⁻⁵ | 1.1 × 10⁻⁵ | 1.5 × 10⁻⁵ |
| Formula 5 | 2.4 × 10⁻⁴ | 6.9 × 10⁻⁵ | 3.8 × 10⁻⁷ | 5.6 × 10⁻⁶ | 1.6 × 10⁻⁵ |
| Formula 6 | 5.4 × 10⁻⁴ | 1.4 × 10⁻⁴ | 2.4 × 10⁻⁶ | 1.1 × 10⁻⁵ | 2.0 × 10⁻⁵ |
| Formula 7 | 4.6 × 10⁻⁴ | 1.5 × 10⁻⁴ | 9.0 × 10⁻⁷ | 6.2 × 10⁻⁵ | 6.1 × 10⁻⁵ |
| Formula 8 | 5.4 × 10⁻⁴ | 1.5 × 10⁻⁴ | 1.5 × 10⁻⁵ | 7.9 × 10⁻⁶ | 4.9 × 10⁻⁵ |

^{a}, ADP; 2 − 5 × 10⁻⁶ M
^{b}, collagen; 2 − 5 × 10⁻⁶ g/ml
^{c}, epinephrine; 1 − 4 × 10⁻⁶ M
^{d}, arachidonic acid; 1 − 4 × 10⁻⁵ M
^{e}, U46619; 1.5 × 10^{-6 M}
^{f}, in the presence of threshold concentration of collagen (1˜8 × 10⁻⁷ g/ml)

### Experimental Example 6

Effect of Enantiomer on the Various Variables in Rat, which Disseminated Intravascular Coagulation (DIC) and Multiple Organ Failure (MOF) Induced by Endotoxin

Rats (Crj:CD(SD); 250±20 g) were used to test sample. Before the compound was administered, the rat was not fed. The compound was orally administered one time per day for two days in an amount of 10 mg˜25 mg/10 ml/kg/day. Distilled water (control group and LPS group) or enantiomer was administered to the rat repeatedly. After one hour, rat was anaesthetized with intramuscular injection of 50 mg/kg of pentobarbital. After 30 min, 15 mg/10 ml/kg of LPS was injected to vein of rat tail for 4 hours by using syringe infusion pump (KDS100, KD scientific, USA). Thereafter, 50 mg/kg of pentobarbital was injected at 2 hour to the rat by intramuscular injection. Therefore, the rat was anaesthetized for all the experiment and operation.

The blood (2 ml for FDP and 6 ml for other variables) was collected from abdominal artery using a plastic syringe, into glass tube containing soy bean trypsin inhibitor and Bothrops atrox venom to measure FDP and into plastic tube containing 2.2% sodium citrate (1/10, volume/volume). The aggregated blood was centrifuged at 1200×g for 5 minutes two times. Before measurement of FDP, the supernatant was stored at freezer for at least 12 hours. Citrated blood was centrifuged at 2000×g for 30 minutes. The supernatant was used for measurement of fibrinogen and PT or APTT time. The aggregated blood collected in glass tube was disposed at the room temperature for 30 min, centrifuged to give the serum for measurement of AST and BUN.

Platelet from total citrated blood was counted by using automatic platelet counter (PLT-4, Texas International Lab.) PT or APTT time was measured by Beckton Dickenson BBL Fibrosystem (Coctkeysville, USA). FDP analysis was accomplished by using Thrombo-wellcotest kit. The said analysis was accomplished by up and down dilution method semi-quantitatively. Measurement of AST and BUN was accomplished by using Autobiochemical analyzer (Hitach 747, Japan).

Conclusion:

As shown in FIG. 2, when LPS was administered to the rat for 4 hours using intravenous injection, platelet count in LPS group was reduced to 259±19.5×10⁶/ml from that in normal group of 721±9.6×10⁶/ml. As shown in FIG. 3, fibrinogen concentration in LPS group was reduced to 100±11.4 mg/dl from that in normal group of 246±8.8 mg/dl. As shown in FIG. 4, the serum level of FDP in LPS group was increased to 202±41 μg/ml from that in normal group of 3±1.1 μg/ml. As shown in FIG. 5, PT time in LPS group was elongated to and 25±1.23 second from that in normal group of 15.6±0.41 second. As shown in FIG. 6, APPT time in LPS group was elongated to 38.9±2.84 second from that of normal group of 19.6±0.43 second. As shown in FIG. 7, the serum level of AST in LPS group was increased to 255±20.0 U/l from that in normal group of 164±5.8 U/l. As shown in FIG. 8, the serum level of BUN in LPS group was increased to 28.9±1.16 U/1 from that of normal group of 16.2±0.46 U/1.

Effects of enantiomers, the compounds represented by formulas 3 to 8 were shown in FIG. 2 to 8. Increase of FDP concentration, reduction of blood platelet count, reduction of fibrinogen concentration, elongation of PT or APTT time, or increase of the serum level of AST and BUN induced by LPS injection were inhibited by administration of the compounds represented by formulas 3 to 8. As shown in FIG. 2, platelet concentration from rat to which S-configuration, the compound represented by formula 3 was administered was higher than one from rat to which R-configuration, the compound represented by formula 4 was administered. Platelet concentration from rat to which S-configuration, the compound represented by formula 5 was administered in an amount of 10 mg/kg was 20% higher than that from rat to which R-configuration, the compound represented by formula 6 was administered in the same amount. Also, platelet concentration from rat to which S-configuration, the compound represented by formula 7 or R-configuration, the compound represented by formula 8 was administered was higher than one from rat to which LPS only was administered, where there is no large difference in the platelet concentration between R- and S-configurations. As shown in FIG. 3, the serum level of fibrinogen from rat to which S-configuration, the compound represented by formula 3, 5 or 7 was administered was 20˜120% as high as one from rat to which R-configuration, the compound represented by formula 4, 6 or 8 was administered. As shown in FIG. 4, the serum level of FDP from rat to which S-configuration, the compound represented by formula 3, 5 or 7 was administered in an amount of 10 mg/kg was correspondingly lower than one from rat to which R-configuration, the compound represented by formula 4, 6 or 8 was administered in the same amount. As shown in FIGS. 5 and 6, PT or APTT time from rat to which S-configuration, the compound represented by formula 3, 5 or 7 was administered was 8-16% and 18˜30% respectively shorter than those from rat to which R-configuration, the compound represented by formula 4, 6 or 8 was administered, respectively. As shown in FIG. 7, the serum level of AST from rat to which R-configuration, the compound represented by formula 4 and 6 or S-configuration, the compound represented by formula 3 and was administered was at the normal level, much lower than one from rat to which LPS was administered, where there is no large difference in the platelet concentration between R- and S-configurations. As shown in FIG. 8, the serum level of BUN from rat to which the compound represented by formula 3 or 4 was administered has no difference. However, the serum level of BUN from rat to which S-enantiomer, the compound represented by formula 5 or 7 was administered was 10˜24% lower than those from rat to which R-enantiomer, the compound represented by formula 6 or 8 was administered, respectively.

### Experimental Example 7

Effect of Enantiomer on the Survival in LPS-Injected Rat

Rats (25˜30 g, ICR), originated from NIH cancer institute (Bethesda, Md., USA) were purchased from Korea Test Animal Corp. (Unsung, Korea). 20 mg/kg of LPS was injected to abdominal cavity of the rat. Thereafter, survival was observed every 24 hours for seven days, to investigate effect of enantiomer on the survival in LPS-injected rat. The compound represented by formula 3 to 8 was administered 30 min before the LPS injection.

Conclusion:

As shown in FIG. 9, the survival of the rat to which all the compounds represented by formulas 3 to 8 was administered was higher than one of the rat to which LPS only was injected. In addition, the survival of the rat to which S-enantiomer, the compounds represented by formulas 3, 5 or 7 was administered was higher than one of the rat to which R-enantiomer, the compounds represented by formulas 4, 6 or 8 was administered. The survival of the rat to which S-enantiomer, the compound represented by formula 3, 5 or 7 was administered in an amount of 15 mg/kg, after six days was 80%, 100% or 73%, respectively. However, the survival of the rat to which LPS only was injected was much lower than one of the rat to which all the compounds was administered, respectively. When the compound represented by formula 3 or 5 was administered in an amount of 30 mg/kg than 15 mg/kg, the survival increased. However, when the compound represented by formula 7 was administered in the same amount, the survival decreased by 7%. Also, the survival of the rat to which R-enantiomer, the compound represented by formula 4, 6 or 8 was administered in an amount of 15 mg/kg, after six days was 60%, 73% or 60%, respectively. Also, the survival of the rat to which R-enantiomer, the compound represented by formula 4, 6 or 8 was administered in an amount of 30 mg/kg, after six days was 67%, 93% or 72%, respectively, where the survival is higher than one of the rat to which the compound was administered in an amount of 15 mg/kg. Therefore, S-enantiomer, the compound represented by formula 3, 5 or 7 showed the higher survival than R-enantiomer, the compound represented by formula 4, 6 or 8. When dosage increased from 15 mg/kg to 30 mg/kg, all the compounds, except the compounds represented by formula 7 increased the survival. When the compounds represented by formula 5, of S-enantiomer was administered to the rat in an amount of 15 mg/kg or 30 mg/kg, all the rats were not dead (survival: 100%). Therefore, the compound represented by formula 5 has most excellent effect of all the compounds. However, the survival of the rat to which higenamine (the racemic mixture of the compounds represented by formula 3 and 4), or the racemic mixture of the compounds represented by formula 5 and 6 was administered was 80% or 90%, respectively. (Kang et al., J. Pharmacol. Exp. Ther., 1999, 291, 314-320) (Kang et al., J. Pharmacol. Exp. Ther., 1999, 128, 357-364)<

### Experimental Example 8

Effect of Enantiomer on the Serum Level of Nitrate/Nitrite in LPS-Injected Rat

The rats were divided into three groups; (1) LPS-injected group (LPS was administered to abdominal cavity in an amount of 20 mg/kg), (2) enantiomer-injected group (LPS+the compounds represented by formula 3 to 8 was administered to abdominal cavity) or (3) saline solution-injected group. After administration of LPS, the rats were anaesthetized with pentobarbital. The blood was collected from rats by using heart punching. The plasma level of nitrite was measured by using the nitrate reductase derived from Aspergillus species, where the nitrate was reduced by enzyme. Particularly, the plasma was diluted with distilled water in a ratio of 1:10. Analysis buffering solution (KH₂PO₄ 50 Mm, NADPH 0.6 Mm, FAD 5 Mm and nitrate reductase 10 U/ml, Ph 5.5) was added to the said plasma. Thereafter, the reaction was accomplished at 37° C. for 30 min. The plasma level of nitrite was measured by using sodium nitrite as a standard, and Griess solution. Standard curve to nitrite was calculated from the result derived from incubation in analysis buffering solution.

Conclusion:

As shown in FIG. 10, the plasma level of NOₓ derived from LPS-injected group was 87±8 μM (n=12). The plasma level of NOₓ derived from enantiomer-injected group was decreased, where the plasma level of NOₓ derived from the group which S-enantiomer, the compounds represented by formula 3, 5 or 7 was administered was higher than one derived from the group which R-enantiomer, the compounds represented by formula 4, 6 or 8 was administered. For example, the plasma level of NOₓ derived from the group which the compounds represented by formula 3 or 4 was administered in an amount of 10 mg/kg was 29±3 μM or 85±7 μM. The plasma level of NOₓ derived from saline solution-injected group was 7˜10 μM (n=6). The plasma level of NOₓ derived from the group which the racemic mixture of the compounds represented by formula 3 or 4 was administered in an amount of 10 mg/kg was decreased to 75±4 μM. Hereinafter, S-enantiomer has inhibitory effect on the plasma level of NOₓ, where the effect is stronger than one of R-enantiomer. Racemic mixture has inhibitory effect on the plasma level of NOₓ, where the effect is between one of R-enantiomer and one of S-enantiomer.

### Experimental Example 9

Acute Toxicity in Rat to which the Compound was Administered

The experiment confirming whether the compound represented by formula 1 or 2 has acute toxicity or not was accomplished as shown in the below.

Sprague-Dawley rats (six weeks) were used as test sample. Each group was comprised of two rats. The compound represented by formula 1 to 6 was emulsified in 1 ml of physiological saline solution to give an emulsion. The said emulsion was administered to two rats one times using intravenous injection and intraperitoneal injection. Also, as for the racemic mixture of the compound (S-configuration or R-configuration), the same procedure shown as in the above was accomplished to measure and compare LD₅₀.

As shown in table 5, LD₅₀ of the group to which S-enantiomer, the compound represented by formulas 3, 5 or 7 was administered was 450 mg/kg, 397 mg/kg or 376 mg/kg. Also, S-enantiomer has acute toxicity, less than R-enantiomer, the compound represented by formula 4, 6 or 8. Also, S-enantiomer has acute toxicity, less than the racemic mixture.

TABLE 5

| | LD₅₀ (mg/kg) | |
|---|---|---|
| | Intraperitioneal | Intravenous |
| | injection | injection |
| Formula 3 | 450 | 154 |
| Formula 4 | 262 | 95 |
| Racemic mixture of formula 3 | 280 | 98 |
| and formula 4 | | |
| Formula 5 | 397 | 172 |
| Formula 6 | 230 | 88 |
| Racemic mixture of formula 5 | 242 | 92 |
| and formula 6 | | |
| Formula 7 | 376 | 184 |
| Formula 8 | 214 | 80 |
| Racemic mixture of formula 7 | 220 | 84 |
| and formula 8 | | |

### INDUSTRIAL APPLICABILITY

As described hereinbefore, enantiomers of tetrahydroisoquinoline derivatives according to the present invention can be used for treatment of heart failure, because of concurrently functioning to promote heart stimulation, vasodilation, inhibit platelet aggregation and suppress induction of iNOS. Further, such compounds can be used for treatment of thrombosis by their inhibitory activity versus platelet aggregation (anti-thrombus action), and for inhibition of tissue damage by inhibition of iNOS expression and suppression of NO production. As well, the optically active tetrahydroisoquinoline derivatives have therapeutic effects on septicemia or disseminated intravascular coagulopathy. In particular, S-configuration compounds function to enhance myocardial contractile force and accelerate heart rates, and are superior in all the above mentioned functions to R-configuration compounds, thus having more enhanced functions than conventional racemic mixtures. Different from S-configurations, R-configurations hardly affect myocardial contraction function and heart rates, and are expected to significantly decrease possibility of arrhythmia, despite administration over long-term periods.

What is claimed is:

1. Tetrahydroisoquinoline derivatives represented by the following general formula 1 or 2, pharmaceutically acceptable salts thereof or prodrugs thereof: 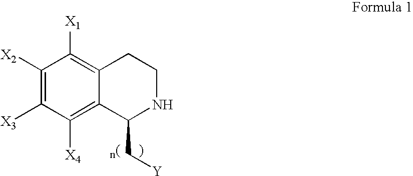
Wherein, X₁, X₂, X₃ and X₄ are independently selected from the group consisting of hydrogen atom, halogen atom, C₁-C₄ alkyl group, hydroxy group and C₁-C₄ alkoxy group, Y represents phenyl group substituted by one or more substituents selected from halogen atom, C₁-C₄ alkyl group, and C₁-C₄ alkoxy group; naphthyl group unsubstituted or substituted by one or more substituents selected from halogen atom, C₁-C₄ alkyl group, hydroxy group and C₁-C₄ alkoxy group; or 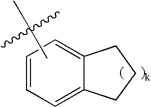
in which, k is an integer of 1 to 3, and n is an integer of 1 to 3. 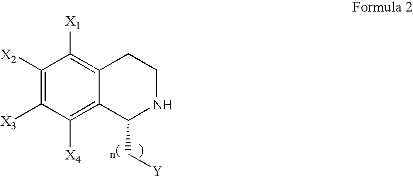
(wherein, X₁, X₂, X₃, X₄, Y and n are as defined in the said formula 1.)

2. Tetrahydroisoquinoline derivatives represented by the following general formula 1 or 2, pharmaceutically acceptable salts thereof or prodrugs thereof according to the claim 1, wherein the derivatives represented by formula 1 or 2 are selected from the group consisting of:
(S)-6,7-Dihydroxy-1-α-naphthylmethyl)-1,2,3,4-tetrahydroisoquinoline, represented by the formula 5;
(R)-6,7-Dihydroxy-1-(α-naphthylmethyl)-1,2,3,4-tetrahydroisoquinoline, represented by the formula 6;
(S)-6,7-Dihydroxy-1-(β-naphthylmethyl)-1,2,3,4-tetrahydroisoquinoline, represented by the formula 7; and
(R)-6,7-Dihydroxy-1-(β-naphthylmethyl)-1,2,3,4-tetrahydroisoquinoline, represented by the formula 8.

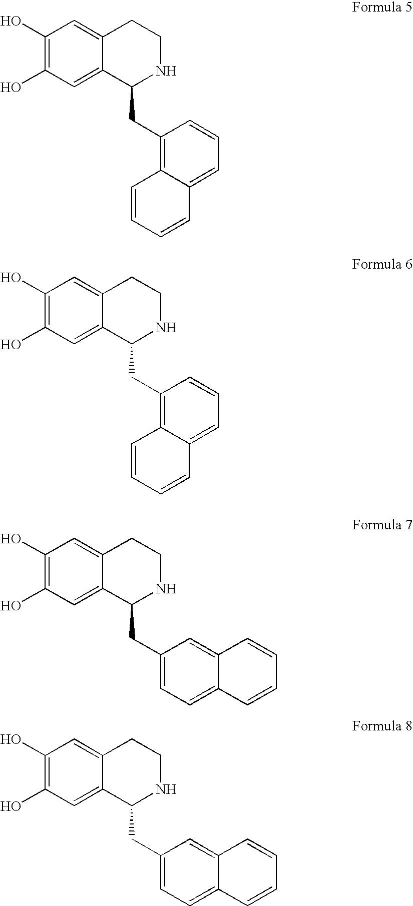

3. Tetrahydroisoquinoline derivatives represented by the following general formula 1 or 2, pharmaceutically acceptable salts thereof or prodrugs thereof according to claim 2, wherein the derivatives represented by formula 1 or 2 are selected from the group consisting of:
(S)-6,7-Dihydroxy-1-(α-naphthylmethyl)-1,2,3,4-tetrahydroisoquinoline, represented by the formula 5; and
(s)-6,7-Dihydroxy-1-(β-naphthylmethyl)-1,2,3,4-tetrahydroisoquinoline, represented by the formula 7.

4. A method for preparing tetrahydroisoquinoline derivatives, comprising the following steps:
condensing p-methoxyphenylacetic acid to 3,4-dimethoxyphenethylamine, to obtain N-(3,4-dimethoxyphenylethyl)(p-methoxyphenyl)acetamide (step 1);
reacting the said compound obtained in the step 1 in the presence of POCl₃ and chloroform, to obtain 6,7-dimethoxy-1-(p-methoxyphenylmethyl)-3,4-dihydroisoquinoline hydrochloride salt (step 2);
reducing the said compound obtained in the step 2 in the presence of (R,R)-Noyori catalyst, to obtain (S)-6,7-dimethoxy-1-(p-methoxyphenylmethyl)-1,2,3,4-tetrahydroisoquinoline, thereafter adding acetic acid and halide acid to the obtained compound, to convert corresponding ammonium salt, and neutralizing the said ammonium salt with basic solution, to obtain corresponding free amine thereof (step 3);
adding acetic acid and halide acid to the said compound obtained in the step 3, to obtain corresponding ammonium salt, (S)-6,7-dimethoxy-1-(p-methoxyphenylmethyl)-1,2,3,4-tetrahydroisoquinoline halide acid salt (step 4);
adding BBr₃ to the said compound obtained in the step 4(demethylation reaction), to obtain (S)-6,7-dihydroxy-1-(p-methoxyphenylmethyl)-1,2,3,4-tetrahydroisoquinoline hydrobromide salt (step 5); and
removing the halide acid salt by neutralizing the said compound obtained in the step 5, to obtain corresponding free amine, (S)-6,7-dihydroxy-1-(p-hydroxyphenylmethyl)-1,2,3,4-tetrahydroisoquinoline (step 6).

5. A method for preparing tetrahydroisoquinoline derivatives, comprising the following steps:
condensing p-methoxyphenylacetic acid to 3,4-dimethoxyphenethylamine, to obtain N-(3,4-dimethoxyphenylethyl)(p-methoxyphenyl)acetamide (step 1);
reacting the said compound obtained in the step 1 in the presence of POCl₃ and chloroform, to obtain 6,7-dimethoxy-1-(p-methoxyphenylmethyl)-3,4-dihydroisoquinoline hydrochloride salt (step 2);
reducing the said compound obtained in the step 2 in the presence of (S,S)-Noyori catalyst, to obtain (R)-6,7-dimethoxy-1-(p-methoxyphenylmethyl)-1,2,3,4-tetrahydroisoquinoline, thereafter adding acetic acid and halide acid to the obtained compound, to convert corresponding ammonium salt, and neutralizing the said ammonium salt with basic solution, to obtain corresponding free amine thereof (step 3);
adding acetic acid and halide acid to the said compound obtained in the step 3, to obtain corresponding ammonium salt, (R)-6,7-dimethoxy-1-(p-methoxyphenylmethyl)-1,2,3,4-tetrahydroisoquinoline halide acid salt (step 4);
adding BBr₃ to the said compound obtained in the step 4(demethylation reaction), to obtain (R)-6,7-dihydroxy-1-(p-hydroxyphenylmethyl)-1,2,3,4-tetrahydroisoquinoline hydrobromide salt (step 5); and
removing. the halide acid salt by neutralizing the said compound obtained in the step 5, to obtain corresponding free amine, (R)-6,7-dihydroxy-1-(p-hydroxyphenylmethyl)-1,2,3,4-tetrahydroisoquinoline (step 6).

6. A method for preparing tetrahydroisoquinoline derivatives, comprising the following steps:
condensing α-naphthylacetic acid to 3,4-dimethoxyphenethylamine, to obtain N-(3,4-dimethoxyphenylethyl) (α-naphthyl)acetamide (step 1);
reacting the said compound obtained in the step 1 in the presence of POCl₃ and chloroform, to obtain 6,7-dimethoxy-1-(α-naphthylmethyl)-3,4-dihydroisoquinoline hydrochloride salt (step 2);
reducing the said compound obtained in the step 2 in the presence of (R,R)-Noyori catalyst, to obtain (S)-6,7-dimethoxy-1-(α-naphthylmethyl)-1,2,3,4tetrahydroisoquinoline, thereafter adding acetic acid and halide acid to the obtained compound, to convert corresponding ammonium salt, and neutralizing the said ammonium salt with basic solution, to obtain corresponding free amine thereof (step 3);
adding acetic acid and halide acid to the said compound obtained in the step 3, to obtain corresponding ammonium salt, (S)-6,7-dimethoxy-1-(α-naphthylmethyl)-1,2,3,4-tetrahydroisoquinoline halide acid salt (step 4);
adding BBr₃ to the said compound obtained in the step 4(demethylation reaction), to obtain (S)-6,7-dihydroxy-1-(α-naphthylmethyl)-1,2,3,4-tetrahydroisoquinoline hydrobromide salt (step 5); and
removing the halide acid salt by neutralizing the said compound obtained in the step 5, to obtain corresponding free amine, (S)-6,7-dihydroxy-1-(α-naphthylmethyl)-1,2,3,4-tetrahydroisoquinoline (step 6).

7. A method for preparing tetrahydroisoquinoline derivatives, comprising the following steps:
condensing α-naphthylacetic acid to 3,4-dimethoxyphenethylamine, to obtain N-(3,4-dimethoxyphenylethyl) (α-naphthyl) acetamide (step 1);
reacting the said compound obtained in the step 1 in the presence of POCl₃ and chloroform, to obtain 6,7-dimethoxy-1-(α-naphthylmethyl)-3,4-dihydroisoquinoline hydrochloride salt (step 2);
reducing the said compound obtained in the step 2 in the presence of (S,S)-Noyori catalyst, to obtain (R)-6,7-dimethoxy-1-(α-naphthylmethyl)-1,2,3,4-tetrahydroisoquinoline, thereafter adding acetic acid and halide acid to the obtained compound, to convert corresponding ammonium salt, and neutralizing the said ammonium salt with basic solution, to obtain corresponding free amine thereof (step 3);
adding acetic acid and halide acid to the said compound obtained in the step 3, to obtain corresponding ammonium salt, (R)-6,7-dimethoxy-1-(α-naphthylmethyl)-1,2,3,4-tetrahydroisoquinoline halide acid salt (step 4);
adding BBr₃ to the said compound obtained in the step 4(demethylation reaction), to obtain (R)-6,7-dihydroxy-1-(α-naphthylmethyl)-1,2,3,4-tetrahydroisoquinoline hydrobromide salt (step 5); and
removing the halide acid salt by neutralizing the said compound obtained in the step 5, to obtain corresponding free amine, (R)-6,7-dihydroxy-1-(α-naphthylmethyl)-1,2,3,4-tetrahydroisoquinoline (step 6).

8. A method for preparing tetrahydroisoquinoline derivatives, comprising the following steps:
condensing β-naphthylacetic acid to 3,4-dimethoxyphenethylamine, to obtain N-(3,4-dimethoxyphenylethyl) (β-naphthyl) acetamide (step 1);
reacting the said compound obtained in the step 1 in the presence of POCl₃ and chloroform, to obtain 6,7-dimethoxy-1-(β-naphthylmethyl)-3,4-dihydroisoquinoline hydrochloride salt (step 2);
reducing the said compound obtained in the step 2 in the presence of (R,R)-Noyori catalyst, to obtain (S)-6,7-dimethoxy-1-(β-naphthylmethyl)-1,2,3,4-tetrahydroisoquinoline, thereafter reacting acetic acid and halide acid to the obtained compound, to convert corresponding ammonium salt, and neutralizing the said ammonium salt with basic solution, to obtain corresponding free amine thereof (step 3);
adding acetic acid and halide acid to the said compound obtained in the step 3, to obtain corresponding ammonium salt, (S)-6,7-dimethoxy-1-(β-naphthylmethyl)-1,2,3,4-tetrahydroisoquinoline halide acid salt (step 4);
adding BBr₃ to the said compound obtained in the step 4(demethylation reaction), to obtain (S)-6,7-dihydroxy-1-(β-naphthylmethyl)-1,2,3,4-tetrahydroisoquinoline hydrobromide salt (step 5); and
removing the halide acid salt by neutralizing the said compound obtained in the step 5, to obtain corresponding free amine, (S)-6,7-dihydroxy-1-(β-naphthylmethyl)-1,2,3,4-tetrahydroisoquinoline (step 6).

9. A method for preparing tetrahydroisoquinoline derivatives, comprising the following steps:
condensing β-naphthylacetic acid to 3,4dimethoxyphenethylamine, to obtain N-(3,4-dimethoxyphenylethyl)(β-naphthyl)acetamide (step 1);
reacting the said compound obtained in the step 1 in the presence of POCl₃ and chloroform, to obtain 6,7-dimethoxy-1-(β-naphthylmethyl)-3,4-dihydroisoquinoline hydrochloride salt (step 2);
reducing the said compound obtained in the step 2 in the presence of (S,S)-Noyori catalyst, to obtain (R)-6,7-dimethoxy-1-(β-naphthylmethyl)-1,2,3,4-tetrahydroisoquinoline, thereafter adding acetic acid and halide acid to the obtained compound, to convert corresponding ammonium salt, and neutralizing the said ammonium salt with basic solution, to obtain corresponding free amine thereof (step 3);
adding acetic acid and halide acid to the said compound obtained in the step 3, to obtain corresponding ammonium salt, (R)-6,7-dimethoxy-1-(β-naphthylmethyl)-1,2,3,4-tetrahydroisoquinoline halide acid salt (step 4);
adding BBr₃ to the said compound obtained in the step 4(demethylation reaction), to obtain (R)-6,7-dihydroxy-1-(β-naphthylmethyl)-1,2,3,4-tetrahydroisoquinoline hydrobromide salt (step 5); and
removing the halide acid salt by neutralizing the said compound obtained in the step 5, to obtain corresponding free amine, (R)-6,7-dihydroxy-1-(β-naphthylmethyl)-1,2,3,4-tetrahydroisoquinoline (step 6).

10. A pharmaceutical composition for treatment of heart failure, comprising a tetrahydroisoquinoline derivative selected from (S)-6,7-dihydroxy-1-(p-hydroxyphenylmethyl)-1,2,3,4-tetrahydroisoquinoline (formula 3); (R)-6,7-dihydroxy-1-(p-hydroxyphenylmethyl)-1,2,3,4-tetrahydroisoquinoline (formula 4); (S)-6,7-dihydroxy-1-α-naphthylmethyl)-1,2,3,4-tetrahydroisoquinoline (formula 5); (R)-6,7-dihydroxy-1-(α-naphthylmethyl)-1,2,3,4-tetrahydroisoquinoline (formula 6); (S)-6,7-dihydroxy-1-(β-naphthylmethyl)-1,2,3,4-tetrahydroisoquinoline (formula 7); and (R)-6,7-dihydroxy-1-(β-naphthylmethyl)-1,2,3,4-tetrahydroisoquinoline (formula 8), pharmaceutically acceptable salt thereof, or prodrug thereof, as an effective ingredient.

11. The composition according to claim 10, for preventing, inhibiting or treating heart failures caused by decrease of myocardial contractile force due to congestive heart failure; ischemic heart diseases; iNOS increase in chronic inflammation; and circulatory disorders by continuous hypertension, arteriosclerosis and coronary artery diseases.

12. A pharmaceutical composition for treatment of thrombosis, comprising a tetrahydroisoquinoline derivative selected from (S)-6,7-dihydroxy-1-(p-hydroxyphenylmethyl)-1,2,3,4-tetrahydroisoquinoline (formula 3); (R)-6,7-dihydroxy-1-(p-hydroxyphenylmethyl)-1,2,3,4-tetrahydroisoquinoline (formula 4); (S)-6,7-dihydroxy-1-(α-naphthylmethyl)-1,2,3,4-tetrahydroisoquinoline (formula 5); (R)-6,7-dihydroxy-1-α-naphthylmethyl)-1,2,3,4-tetrahydroisoquinoline (formula 6); (S)-6,7-dihydroxy-1-(β-naphthylmethyl)-1,2,3,4-tetrahydroisoquinoline (formula 7); and (R)-6,7-dihydroxy-1-(β-naphthylmethyl)-1,2,3,4-tetrahydroisoquinoline (formula 8), pharmaceutically acceptable salt thereof, or prodrug thereof, as an effective ingredient.

13. The composition according to claim 12, for preventing, inhibiting or treating thrombogenesis in ischemic cerebral vascular disorders, coronary artery diseases, ischemic myocardial infarction, chronic arterial obstruction, thrombosis or embolism after surgery, induced by thrombus.

14. A pharmaceutical composition for treatment of inflammation, comprising a tetrahydroisoquinoline derivative selected from (S)-6,7-dihydroxy-1-(p-hydroxyphenylmethyl)-1,2,3,4-tetrahydroisoquinoline (formula 3); (R)-6,7-dihydroxy-1-(p-hydroxyphenylmethyl)-1,2,3,4-tetrahydroisoquinoline (formula 4); (S)-6,7-dihydroxy-1-(α-naphthylmethyl)-1,2,3,4-tetrahydroisoquinoline (formula 5); (R)-6,7-dihydroxy-1-(α-naphthylmethyl)-1,2,3,4-tetrahydroisoquinoline (formula 6); (S)-6,7-dihydroxy-1-(β-naphthylmethyl)-1,2,3,4-tetrahydroisoquinoline (formula 7); and (R)-6,7-dihydroxy-1-(β-naphthylmethyl)-1,2,3,4-tetrahydroisoquinoline (formula 8), pharmaceutically acceptable salt thereof, or prodrug thereof, as an effective ingredient.

15. The composition according to claim 14, for preventing, inhibiting or treating inflammatory diseases caused by tissue or organ damages and ischemia and reperfusion injuries caused by arteriosclerosis, myocardial infarction and cerebral apoplexy.

16. A pharmaceutical composition for treatment of septicemia, comprising a tetrahydroisoquinoline derivative selected from (S)-6,7-dihydroxy-1-(p-hydroxyphenylmethyl)-1,2,3,4-tetrahydroisoquinoline (formula 3); (R)-6,7-dihydroxy-1-(p-hydroxyphenylmethyl)-1,2,3,4-tetrahydroisoquinoline (formula 4); (S)-6,7-dihydroxy-1-α-naphthylmethyl)-1,2,3,4-tetrahydroisoquinoline (formula 5); (R)-6,7-dihydroxy-1-(α-naphthylmethyl)-1,2,3,4-tetrahydroisoquinoline (formula 6); (S)-6,7-dihydroxy-1-(β-naphthylmethyl)-1,2,3,4-tetrahydroisoquinoline (formula 7); and (R)-6,7-dihydroxy-1-(β-naphthylmethyl)-1,2,3,4-tetrahydroisoquinoline (formula 8), pharmaceutically acceptable salt thereof, or prodrug thereof, as an effective ingredient.

17. The composition according to claim 16, for treating septicemia caused by multiple organ failure and disseminated intravascular coagulation.

18. A pharmaceutical composition for treatment of disseminated intravascular coagulopathy, comprising a tetrahydroisoquinoline derivative selected from (S)-6,7-dihydroxy-1-(p-hydroxyphenylmethyl)-1,2,3,4-tetrahydroisoquinoline (formula 3); (R)-6,7-dihydroxy-1-(p-hydroxyphenylmethyl)-1,2,3,4-tetrahydroisoquinoline (formula 4); (S)-6,7-dihydroxy-1-α-naphthylmethyl)-1,2,3,4-tetrahydroisoquinoline (formula 5); (R)-6,7-dihydroxy-1-(α-naphthylmethyl)-1,2,3,4-tetrahydroisoquinoline (formula 6); (S)-6,7-dihydroxy-1-(β-naphthylmethyl)-1,2,3,4-tetrahydroisoquinoline (formula 7); and (R)-6,7-dihydroxy-1-(β-naphthylmethyl)-1,2,3,4-tetrahydroisoquinoline (formula 8), pharmaceutically acceptable salt thereof, or prodrug thereof, as an effective ingredient.

19. The composition according to claim 18, for treating disseminated intravascular coagulopathy caused by drastically decreased platelet number, bleeding, shock, thrombus, vascular obstruction due to activation of rapid blood coagulation.
